(19) United States
(12) Patent Application Publication
(10) Pub. No.: US 2004/0010038 A1
(43) Pub. Date: Jan. 15, 2004

(54) Propargylamino indan derivatives and propargylamino tetralin derivatives as brain-selective MAO inhibitors

(76) Inventors: Eran Blaugrund, Rehovot (IL); Yaacov Herzig, Ra'ananna (IL); Jeffrey Sterling, Jerusalem (IL)

Correspondence Address: John P. White, Cooper & Dunham LLP, 1185 Avenue of the Americas, New York, NY, 10036, US

(21) Appl. No.: 10/375,369

(22) Filed: Feb. 27, 2003

Related U.S. Application Data
(60) Provisional application No. 60/360,265, filed on Feb. 27, 2002.

(51) Int. Cl. A61K 31/375; C07D 307/62; A61K 31/24

(52) U.S. Cl. 514/540; 514/474; 560/136; 549/316

(57) ABSTRACT

The subject invention provides derivatives of propargylamino indan (PAI) and propargylamino tetralin that selectively inhibit monoamine oxidase (MAO) in the brain, having the structure:

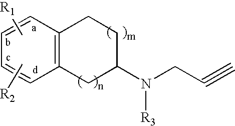

wherein R₁ is OC(O)R₉ and R₂ is H,

wherein R₉ is branched or unbranched C₁ to C₆ alkyl, aryl, or aralkyl, or

R₁ is OC(O)R₄ and R₂ is OC(O)R₄,

wherein R₄ is branched or unbranched C₁ to C₆ alkyl, aryl, aralkyl or NR₅R₆,

wherein R₅ and R₆ are each independently H, C₁ to C₈ alkyl, C₆ to C₁₂ aryl, C₆ to C₁₂ aralkyl or C₆ to C₁₂ cycloalkyl, each optionally substituted;

wherein R₃ is H or C₁ to C₆ alkyl;

wherein n is 0 or 1; and

wherein m is 1 or 2,

or a pharmaceutically acceptable salt thereof. Additionally, the subject invention provides methods of treating neurological disorders using these compounds, uses of these compounds for the manufacture of medicaments for treating neurological disorders and processes for synthesis of these compounds.

This application claims the benefit of U.S. Provisional Application No. 60/360,265, filed Feb. 27, 2002, the contents of which are hereby incorporated by reference.

Throughout this application, various references are referenced by short citations within parenthesis. Full citations for these references may be found at the end of the specification, immediately preceding the claims. These references, in their entireties, are hereby incorporated by reference to more fully describe the state of the art to which this invention pertains.

### FIELD OF THE INVENTION

The subject of this invention provides for derivatives of propargylaminoindans and propargylaminotetralins that are irreversible inhibitors of the enzyme monoamine oxidase A and/or B and also for prodrugs for the administration of these compounds. Such compounds may be useful in the treatment of Parkinson's disease, Alzheimer's disease, depression and other neurological disorders.

### BACKGROUND OF THE INVENTION

The enzyme monoamine oxidase (MAO) plays an essential role in the metabolic degradation of important amine neurotransmitters including dopamine, serotonin and noradrenaline. Thus, agents that inhibit MAO are of potential therapeutic benefit for a variety of neurological disease indications, including Parkinson's disease, Alzheimer's disease, depression, epilepsy, narcolepsy, amyotrophic lateral sclerosis (ALS), etc. (Szelnyi, I.; Bentue-Ferrer et al.; Loscher et al.; White et al.; U.S. Pat. No. 5,744,500). Other diseases and conditions which have been associated with toxic levels of monoamine oxidase-B are memory disorders (The interaction of L-deprenyl and scopolamine on spatial learning/memory in rats), panic, post-traumatic stress disorder (PTSD), sexual dysfunction, attention deficit and hyperactivity syndrome (ADHD) (Potential applications for monoamine oxidase B inhibitors), attention deficit disorder (Kleywegt), and Tourette's syndrome (Treatment of Tourette's: Overview).

Many inhibitors of MAO are chiral molecules (U.S. Pat. No. 5,744,500). Although one enantiomer often shows some stereoselectivity in relative potency towards MAO-A and -B, a given enantiomeric configuration is not always more selective than its isomer in discriminating between MAO-A and -B (Hazelhoff et al., Naunyn-Schmeideberg's Arch. Pharmacol.).

MAO inhibitors can also be classified as reversible inhibitors which inhibit the enzyme by a competitive mechanism or as irreversible inhibitors which are generally mechanism based (suicide inhibitors) (Dostert). For example, moclobemide is a reversible MAO-A-specific inhibitor (Fitton et al.) developed as an anti-depressant. Likewise, rasagiline (U.S. Pat. No. 5,744,500) and selegiline (Chrisp et al.) are MAO-B-selective irreversible inhibitors.

Irreversible inhibitors have the advantage of lower, less frequent dosing since their MAO inhibition is not based directly on the drugs' pharmacokinetic behavior, but rather on the de novo regeneration of the MAO enzyme.

MAO also plays an essential role in the oxidative deamination of biogenic and food-derived amines, both in the central nervous system and in peripheral tissues. MAO is found in two functional isoenzyme forms, MAO-A and MAO-B, each of which shows preferential affinity for substrates and specificity toward inhibitors. Thus, MAO-A preferentially oxidizes serotonin, noradrenaline and adrenaline, whereas MAO-B preferentially metabolizes phenylethylamine. Dopamine is a substrate for both forms of the enzyme(Szelenyi, I.).

N-Propargyl-(1R)-aminoindan is known to be a potent B-selective inhibitor of MAO (U.S. Pat. No. 5,457,133). Various derivatives of this compound have been prepared and shown to have varying degrees of potency and selectivity for the inhibition of MAO-A and/or -B. There is no currently accepted theory explaining the effect of structure on the activity (SAR) of the various substituted propargylaminoindans.

The dopamine agonistic activity and MAO inhibitory properties of 7-(methyl-prop-2-ynylamino)-tetralin-2-ol and 7-(methyl-prop-2-ynylamino)-tetralin-2,3-diol have been reported (Hazelhoff et al., Eur. J. Pharmacol.). The details of the synthesis of these compounds have not been published, however.

6,7-di-O-benzoyl-2-aminotetralin has been reported as a prodrug of the dopaminergic agonist 6,7-di-hydroxy-2-aminotetralin (Horn et al.). However, no N-propargyl derivatives were reported and the compounds were not shown to have MAO inhibitory or neuroprotective activities.

7-(propyl-prop-2-ynylamino)-tetralin-2-ol has been reported as an intermediate in the preparation of 7-[(3-iodoallyl)-propylamino]-tetralin-2-ol. Only the latter has been pharmacologically characterized as D₃-dopamine receptor ligand (Chumpradit et al.). No other N-alkyl substituents were described.

Florvall et al. report the preparation of amino acid-based prodrugs of amiflamine analogues. Amiflamine is a reversible MAO-A inhibitor.

PCT International Application No. PCT/US97/24155 concerns carbamate aminoindan derivatives, including propargylamines, as inhibitors of MAO-A and MAO-B for the treatment of Alzheimer's disease and other neurological conditions. However, the compounds of PCT/US97/24155 are not selective for MAO over acetylcholinesterase (“AChE”). Thus, the compounds generally inhibit acetylcholinesterase along with MAO. Acetylcholinesterase inhibition is a route implicated in certain neurological disorders, but is a different route from the route of MAO inhibition.

U.S. Pat. No. 6,303,650 discloses derivatives of 1-aminoindan as selective MAO B inhibitors that additionally inhibit acetylcholinesterase. The reference teaches that its compounds can be used to treat depression, Attention Deficit Disorder (ADC), Attention Deficit and Hyperactivity Disorder (ADHD), Tourette's Syndrome, Alzheimer's Disease and other dementias such as senile dementia, dementia of the Parkinson's type, vascular dementia and Lewy body dementia.

Many irreversible MAO inhibitors contain the propargyl amine functionality. This pharmacophore is responsible for the MAO inhibitory-activity of such compounds. Some propargylamines have been shown to have neuroprotective/neurorescue properties independent of their MAO inhibition activity (U.S. Pat. No. 4,844,033; Krageten et al.).

PCT International Application No. PCT/IL96/00115 relates to pharmaceutical compositions comprising racemic, (S), and (R)—N-propargyl-1-aminoindan. (R)—N-propargyl-1-aminoindan selectively inhibits MAO-B in the treatment of Parkinson's disease and other neurological disorders(PCT/IL96/00115).

Derivatives of 1-aminoindan, including propargyl aminoindan, and their salts are described in many U.S. patents (U.S. Pat. Nos. 5,639,913, 5,877,221, 5,880,159, 5,877,218, 5,914,349, 5,994,408) and a PCT International Application (PCT/US95/00245). These references disclose racemic, R and S enantiomers of 1-aminoindan derivatives for the treatment of Parkinson's disease and other neurological conditions (U.S. Pat. Nos. 5,639,913, 5,877,221, 5,880,159, 5,877,218, 5,914,349, 5,994,408, PCT/US95/00245).

PCT International Application No. PCT/US97/24155 concerns aminoindan derivatives, including propargyl aminoindan, as inhibitors of MAO-A and MAO-B for the treatment of Parkinson's disease and other neurological conditions. The publication reveals that the disclosed compounds exhibit a greater selectivity for MAO-A and MAO-B in the brain than in the liver or intestine.

U.S. Pat. No. 6,316,504 discloses that the R(+) enantiomer of N-propargyl-1-aminoindan is a selective irreversible inhibitor of MAO-B. The patent indicates that (R)—N-propargyl-1-aminoindan is useful for the treatment of Parkinson's disease, a memory disorder, dementia, depression, hyperactive syndrome, an affective illness, a neurodegenerative disease, a neurotoxic injury, stroke, brain ischemia, a head trauma injury, a spinal trauma injury, neurotrauma, schizophrenia, an attention deficit disorder, multiple sclerosis, and withdrawal symptoms.

European Patent No. 436492 discloses the R enantiomer of N-propargyl-1-aminoindan as a selective irreversible inhibitor of MAO-B in the treatment of Parkinson's disease and other neurological conditions. Numerous U.S. patents also relate to the MAO B inhibition of (R)—N-propargyl-1-aminoindan and its use for treating patients suffering from Parkinson's Disease and other neurological disorders (U.S. Pat. Nos. 5,387,612, 5,453,446, 5,457,133, 5,519,061, 5,532,415, 5,576,353, 5,668,181, 5,744,500, 5,786,390 and 5,891,923).

PCT International Application No. PCT/IL97/00205 discloses S-(−)-N-propargyl-1-aminoindan or a pharmaceutically acceptable salt thereof for the treatment of a neurological disorder of neurotrauma or for improving memory. The compounds were found to be neuroprotective, but not inhibitory of MAO-A or MAO-B (PCT/IL97/00205).

U.S. Pat. No. 5,486,541 provides N-propargyl-1-amonoindan monofluorinated in the phenyl ring as selective inhibitors of MAO-B. These compounds are presented as useful in the treatment of Parkinson's disease, memory disorders, dementia of the Alzheimer's type, depression and the hyperactive syndrome in children.

Among the many derivatives of propargylaminoindan mentioned in the prior art are hydroxy-propargylaminoindans. U.S. Pat. No. 3,513,244 lists some racemic N-propargylamino indanols and tetralinols for use as antihypertensives. These compounds are not exemplified chemically and are not pharmacologically characterized (U.S. Pat. No. 3,513,244).

N-propargylamino indanol also appears in E.P. 267024 as a hydrofluorene derivative, i.e., 3-amino-4-indanol (7-OH fluorene). The hydrofluorene derivatives and salts in E.P. 267024 are employed as cerebral activators in the treatment of anoxemia and hypoxemia. In addition, such derivatives help prevent arrhythmia and heart failure caused by lack of oxygen (E.P. 267024). The derivatives also act as antioxidants and cholinergic nerve system activating agents (E.P. 267024).

### SUMMARY OF THE INVENTION

The subject invention provides a compound having the structure:

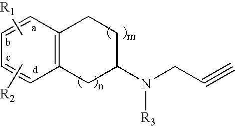

wherein R₁ is OC(O)R₉ and R₂ is H,

wherein R₉ is branched or unbranched C₁ to C₆ alkyl, aryl, or aralkyl, or

R₁ is OC(O)R₄ and R₂ is OC(O)R₄,

wherein R₄ is branched or unbranched C₁ to C₆ alkyl, aryl, aralkyl or NR₅R₆,

wherein R₅ and R₆ are each independently H, C₁ to C₈ alkyl, C₆ to C₁₂ aryl, C₆ to C₁₂ aralkyl or C₆ to C₁₂ cycloalkyl, each optionally substituted;

wherein R₃ is H or C₁ to C₆ alkyl;

wherein n is 0 or 1; and

wherein m is 1 or 2,

or a pharmaceutically acceptable salt thereof.

The subject invention also provides a compound having the structure:

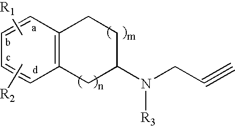

wherein R₁ is OH;

wherein R₂ is H or OC(O)R₄ when R₁ is attached to the “a” carbon or the “d” carbon, or

R₂ is OC(O)R₄ when R₁ is attached to the “b” carbon or the “c” carbon;

wherein R₄ is C₁ to C₆ branched or unbranched alkyl, aryl, aralkyl or NR₅R₆,

wherein R₅ and R₆ are each independently H, C₁ to C₈ alkyl, C₆ to C₁₂ aryl, C₆ to C₁₂ aralkyl or C₆ to C₁₂ cycloalkyl, each optionally substituted;

wherein n is 0 or 1, and m is 1 or 2; and

wherein R₃ is H or Me when n is 1 and m is 1, or R₃ is H or C₁ to C₆ alkyl when n is 0 or m is 2,

or a pharmaceutically acceptable salt thereof.

In addition, the subject invention provides a compound having the structure:

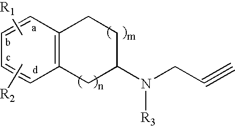

wherein the compound is an optically pure enantiomer;

wherein R₁ is OH;

wherein R₂ is H;

wherein R₃ is H or C₁ to C₆ alkyl;

wherein n is 0 or 1; and

wherein m is 1 or 2,

or a pharmaceutically acceptable salt thereof.

The subject invention further provides a compound having the structure:

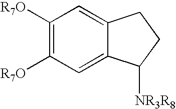

wherein R₇ is H, C₁ to C₆ alkyl, aryl, aralkyl or C(O)R₄,

wherein R₄ is branched or unbranched C₁ to C₆ alkyl, aryl, aralkyl or NR₅R₆,

wherein R₅ and R₆ are each independently H, C₁ to C₈ alkyl, C₆ to C₁₂ aryl, C₆ to C₁₂ aralkyl or C₆ to C₁₂ cycloalkyl, each optionally substituted;

wherein R₃ is H or C₁ to C₆ alkyl;

wherein R₈ is H or t-butoxycarbonyl (Boc).

The subject invention also provides a method of treating a subject afflicted with a neurological disease comprising administering to the subject a compound having the structure:

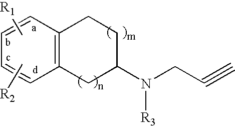

wherein R₁ is OH or OC(O)R₉, and wherein R₉ is branched or unbranched C₁ to C₆ alkyl, aryl, or aralkyl;

R₂ is H or OC(O)R₄, or both R₁ and R₂ are OC(O)R₄,

wherein R₄ is branched or unbranched C₁ to C₆ alkyl, aryl, aralkyl or NR₅R₆,

wherein R₅ and R₆ are each independently H, C₁ to C₈ alkyl, C₆ to C₁₂ aryl, C₆ to C₁₂ aralkyl or C₆ to C₁₂ cycloalkyl, each optionally substituted;

wherein R₃ is H or C₁ to C₆ alkyl;

wherein n is 0 or 1; and

wherein m is 1 or 2,

or a pharmaceutically acceptable salt thereof, or a prodrug which becomes the compound in the subject, so as to thereby treat the neurological disease in the subject.

Furthermore, the subject invention provides a method of treating a subject afflicted with a neurological disease comprising administering to the subject a compound having the structure:

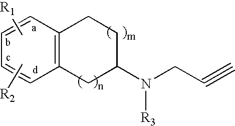

wherein R₁ is OH or OC(O)R₄;

wherein R₂ is H or OC(O)R₄,

wherein R₄ is branched or unbranched C₁ to C₆ alkyl, aryl, aralkyl or NR₅R₆,

wherein R₅ and R₆ are each independently H, C₁ to C₈ alkyl, C₆ to C₁₂ aryl, C₆ to C₁₂ aralkyl or C₆ to C₁₂ cycloalkyl, each optionally substituted;

wherein R₃ is H or C₁ to C₆ alkyl;

wherein n is 0 or 1; and

wherein m is 1 or 2,

or a pharmaceutically acceptable salt thereof, or a prodrug which becomes the compound in the subject, so as to thereby treat the neurological disease in the subject.

The subject invention additionally provides a process for preparing a compound having the structure:

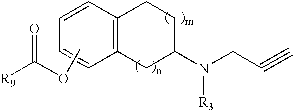

wherein n is 0 or 1, and m is 1 or 2;

wherein R₃ is H or C₁ to C₆ alkyl; and

wherein R₉ is branched or unbranched C₁ to C₆ alkyl, aryl, or aralkyl;

comprising the step of reacting

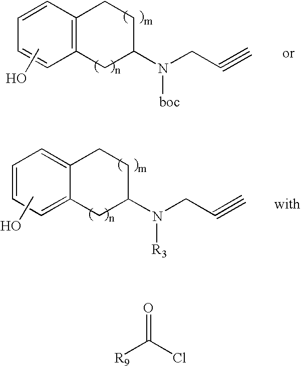

in the presence of an acid or 4-dimethylaminopyridine (DMAP) to form the compound.

The subject invention also provides a process for preparing a compound having the structure:

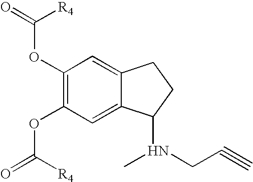

wherein R₄ is branched or unbranched C₁ to C₆ alkyl, aryl, aralkyl or NR₅R₆,

wherein R₅ and R₆ are each independently H, C₁ to C₈ alkyl, C₆ to C₁₂ aryl, C₆ to C₁₂ aralkyl or C₆ to C₁₂ cycloalkyl, each optionally substituted;

which process comprises:

(a) reacting a compound having the structure:

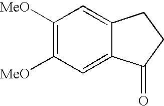

with AlCl₃ or BBr₃ in the presence of toluene to produce a compound having the structure:

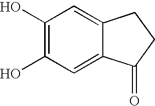

(b) reacting the product formed in step (a) with benzyl chloride and K₂CO₃ in the presence of dimethyl formamide (DMF) to produce a compound having the structure:

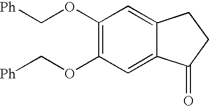

(c) reacting the product formed in step (b) with MeNH₂.HCl, NaCNBH₃ in tetrahydrofuran (THF)/MeOH to produce a compound having the structure:

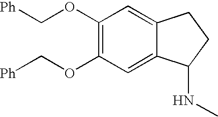

(d) reacting the product formed in step (c) with H₂, Pd/C and MeOH to produce a compound having the structure:

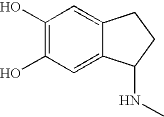

(e) reacting the product formed in step (d) with Boc₂O, dioxane/H₂O and NaHCO₃ to produce a compound having the structure:

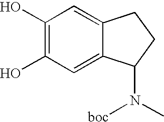

(f) reacting the product formed in step (e) with R₄COCl, Et₃N in CH₂Cl₂ in the presence of 4-dimethylaminopyridine (DMAP) to produce a compound having the structure:

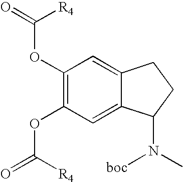

(g) reacting the product formed in step (f) with HCl/dioxane to produce a compound having the structure:

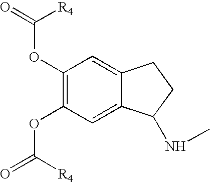

(h) reacting the product formed in step (g) with propargyl bromide, K₂CO₃ in CH₃CN and then with HCl/ether and MeOH to produce a compound having the structure:

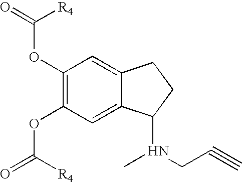

The subject invention also provides the use of a compound or a prodrug of a compound which becomes the compound having the structure:

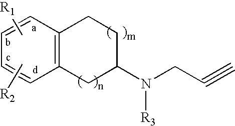

wherein R₁ is OH or OC(O)R₄;

wherein R₂ is H, OH or OC(O)R₄,

wherein R₄ is branched or unbranched C₁ to C₆ alkyl, aryl, aralkyl or NR₅R₆,

wherein R₅ and R₆ are each independently H, C₁ to C₈ alkyl, C₆ to C₁₂ aryl, C₆ to C₁₂ aralkyl or C₆ to C₁₂ cycloalkyl, each optionally substituted;

wherein R₃ is H or C₁ to C₆ alkyl;

wherein n is 0 or 1; and

wherein m is 1 or 2,

or a pharmaceutically acceptable salt thereof, for the manufacture of a medicament for treating a subject afflicted with a neurological disease, wherein the compound is to be periodically administered to the subject in a therapeutically effective dose.

Additionally, the subject invention provides the use of a compound or a prodrug of a compound which becomes the compound having the structure:

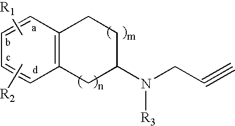

wherein R₁ is OH or OC(O)R₉, and

wherein R₉ is branched or unbranched C₁ to C₆ alkyl, aryl, or aralkyl;

R₂ is H or OC(O)R₄, or both R₁ and R₂ are OC(O)R₄,

wherein R₄ is C₁ to C₆ alkyl, aryl, aralkyl or NR₅R₆,

wherein R₅ and R₆ are each independently H, C₁ to C₈ alkyl, C₆ to C₁₂ aryl, C₆ to C₁₂ aralkyl or C₆ to C₁₂ cycloalkyl, each optionally substituted;

wherein R₃ is H or C₁ to C₆ alkyl;

wherein n is 0 or 1; and

wherein m is 1 or 2,

or a pharmaceutically acceptable salt thereof, for the manufacture of a medicament for treating neurological disease in a subject, wherein the compound is to be periodically administered to the subject in a therapeutically effective dose.

### DESCRIPTION OF THE DRAWINGS

FIG. 1 presents routes for the manufacture of compounds with the following structures:

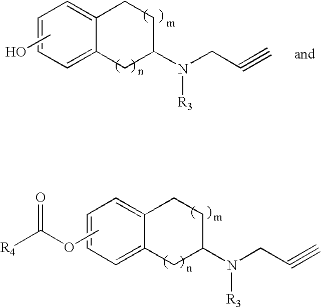

FIG. 2 displays routes for the manufacture of a compound with the following structure:

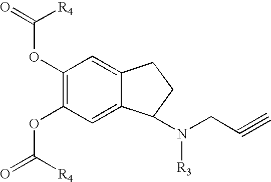

In FIG. 2, the letters a)-i) are used to designate the following: a) AlCl₃, toluene; b) BnCl, K₂CO₃, DMF; c) R₃—NH₂, HCl, NaCNBH₃, THF/MeOH; d) H_{2,} Pd/C, MeOH; e) Boc₂O, dioxane/H₂O, NaHCO₃; f) R₄—COCl, Et₃N, DMAP, CH₂Cl₂; g) HCl/dioxane; h) propargyl bromide, K₂CO₃, CH₃CN; and i) HCl/ether, MeOH.

FIG. 3 depicts routes for the manufacture of compounds with the structures:

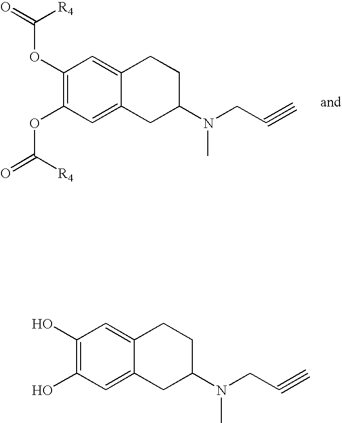

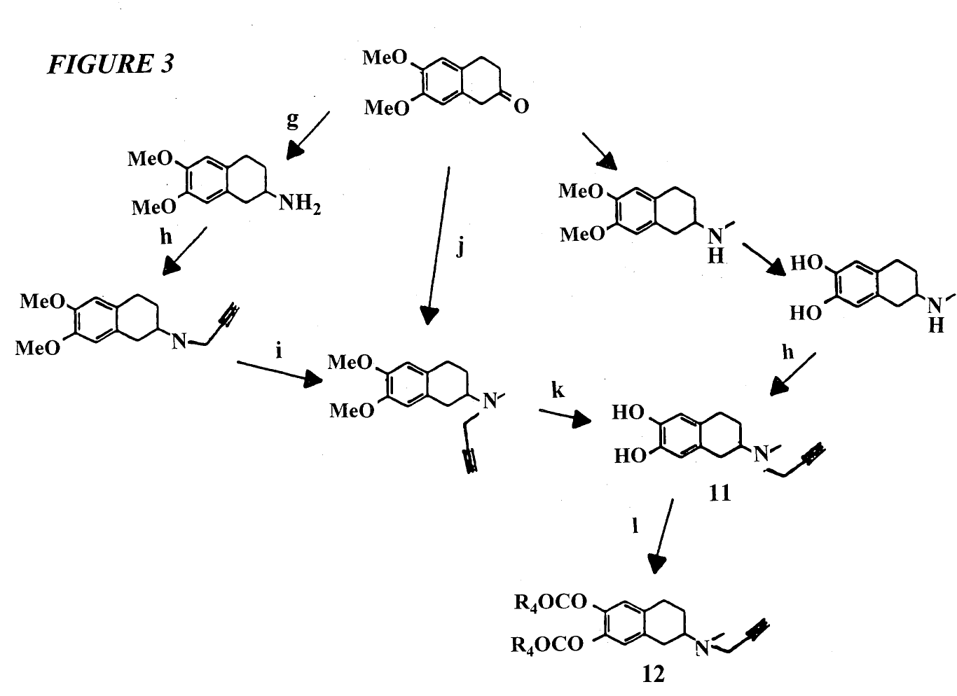

In FIG. 3, the letters g)-l) are used to designate the following: g) NaCNBH₃, NH₄OAc; h) propargyl bromide, ACN, K₂CO₃; i) NaCNBH₃, paraformaldehyde; j) N-methylpropargylamine, NaCNBH₃; k) BBr₃; and l) R₄COCl, TFA or DMAP.

### DETAILED DESCRIPTION OF THE INVENTION

The subject invention provides a compound having the structure:

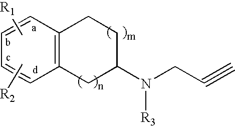

wherein R₁ is OC(O)R₉ and R₂ is H,

wherein R₉ is branched or unbranched C₁ to C₆ alkyl, aryl, or aralkyl, or

R₁ is OC(O)R₄ and R₂ is OC(O)R₄,

wherein R₄ is branched or unbranched C₁ to C₆ alkyl, aryl, aralkyl or NR₅R₆,

wherein R₅ and R₆ are each independently H, C₁ to C₈ alkyl, C₆ to C₁₂ aryl, C₆ to C₁₂ aralkyl or C₆ to C₁₂ cycloalkyl, each optionally substituted;

wherein R₃ is H or C₁ to C₆ alkyl;

wherein n is 0 or 1; and

wherein m is 1 or 2,

or a pharmaceutically acceptable salt thereof.

In one embodiment, the pharmaceutically acceptable salt is the acetate salt, mesylate salt, esylate, tartarate salt, hydrogen tartarate salt, benzoate salt, phenylbutyrate salt, phosphate salt, citrate salt, ascorbate salt, mandelate salt, adipate salt, octanoate salt, the myristate salt, the succinate salt, or fumarate salt.

In another embodiment, the compound has the structure:

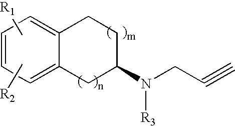

In a further embodiment, the compound has the structure:

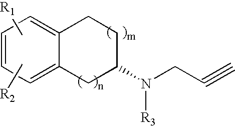

In yet another embodiment, the compound has the structure:

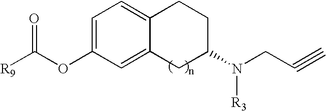

In one embodiment, n is 1.

In a further embodiment, the compound has the structure:

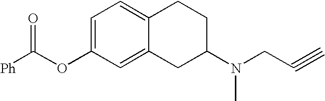

In an added embodiment, n is 0.

In yet another embodiment, the compound has the structure:

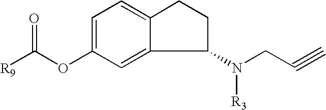

In still another embodiment, the compound has the structure:

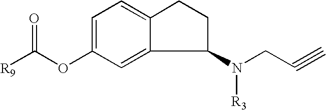

In one embodiment, R₉ is Me and R₃ is H.

In another embodiment, R₉ is tBu and R₃ is H.

In a further embodiment, R₉ is nBu and R₃ is H.

In yet another embodiment, R₉ is CH₂Ph and R₃ is H.

In an additional embodiment, R₉ is Ph and R₃ is H.

In still another embodiment, wherein R₉ is Me and R₃ is Me.

In a further embodiment, R₉ is nBu and R₃ is Me.

In one embodiment, R₉ is Ph and R₃ is Me.

In an added embodiment, R₉ is tBu and R₃ is Me.

In another embodiment, R₉ is Ph(Me) and R₃ is Me.

In still another embodiment, R₉ is Ph(OMe)₂ and R₃ is Me.

In a further embodiment, R₉ is Ph(OMe)₂ and R₃ is H.

In one embodiment, the compound has the structure:

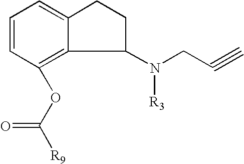

In an additional embodiment, R₃ is Me and R₉ is Me.

In a further embodiment, R₃ is Me and R₉ is Ph.

In another embodiment, R₃ is Me and R₉ is Ph(OMe)₂.

In yet another embodiment, the compound has the structure:

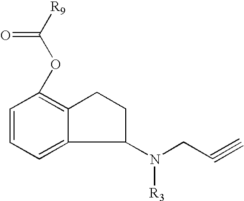

In an added embodiment, R₃ is Me and R₉ is Me.

In still another embodiment, R₃ is H and R₉ is Ph.

In one embodiment, R₃ is H and R₉ is Ph(OMe)₂.

In another embodiment, the compound has the structure:

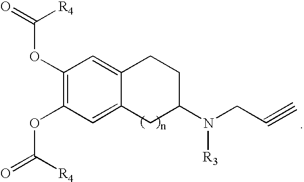

In a further embodiment, n is 0.

In yet another embodiment, R₄ is Ph and R₃ is Me.

In one embodiment, n is 1.

In still another embodiment, R₃ is Me.

In an added embodiment, the compound has the structure:

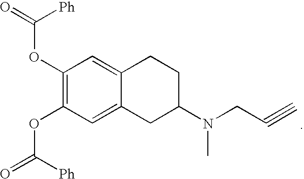

The subject invention also provides a compound having the structure:

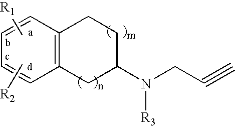

wherein R₁ is OH;

wherein R₂ is H or OC(O)R₄ when R₁ is attached to the “a” carbon or the “d” carbon, or

R₂ is OC(O)R₄ when R₁ is attached to the “b” carbon or the “c” carbon;

wherein R₄ is C₁ to C₆ branched or unbranched alkyl, aryl, aralkyl or NR₅R₆,

wherein R₅ and R₆are each independently H, C₁ to C₈ alkyl, C₆ to C₁₂ aryl, C₆ to C₁₂ aralkyl or C₆ to C₁₂ cycloalkyl, each optionally substituted;

wherein n is 0 or 1, and m is 1 or 2; and

wherein R₃ is H or Me when n is 1 and m is 1, or R₃ is H or C₁ to C₆ alkyl when n is 0 or m is 2,

or a pharmaceutically acceptable salt thereof.

In one embodiment, the pharmaceutically acceptable salt is the acetate salt, mesylate salt, esylate, tartarate salt, hydrogen tartarate salt, benzoate salt, phenylbutyrate salt, phosphate salt, citrate salt, ascorbate salt, mandelate salt, adipate salt, octanoate salt, the myristate salt, the succinate salt, or fumarate salt.

In another embodiment, the compound has the structure:

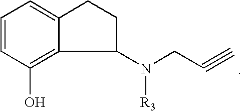

In an additional embodiment, R₃ is H.

In a further embodiment, R₃ is Me.

In yet another embodiment, the compound has the structure:

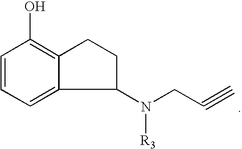

In still another embodiment, R₃ is H.

In one embodiment, R₃ is Me.

In a further embodiment, n is 0.

Additionally, the subject invention provides a compound having the structure:

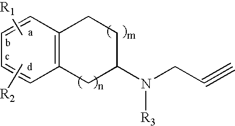

wherein the compound is an optically pure enantiomer;

wherein R₁ is OH;

wherein R₂ is H;

wherein R₃ is H or C₁ to C₆ alkyl;

wherein n is 0 or 1; and

wherein m is 1 or 2,

or a pharmaceutically acceptable salt thereof.

In one embodiment, the pharmaceutically acceptable salt is the acetate salt, mesylate salt, esylate, tartarate salt, hydrogen tartarate salt, benzoate salt, phenylbutyrate salt, phosphate salt, citrate salt, ascorbate salt, mandelate salt, adipate salt, octanoate salt, the myristate salt, the succinate salt, or fumarate salt.

In a further embodiment, the compound has the structure:

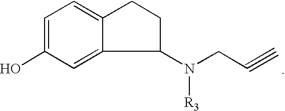

In another embodiment, the compound has the structure:

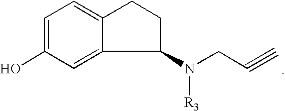

In an added embodiment, R₃ is H.

In yet another embodiment, R₃ is Me.

In a further embodiment, the compound has the structure:

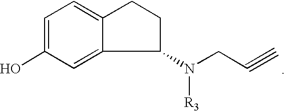

In one embodiment, R₃ is H.

In another embodiment, R₃ is Me.

The subject invention further provides a compound having the structure:

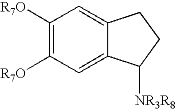

wherein R₇ is H, C₁ to C₆ alkyl, aryl, aralkyl or C(O)R₄,

wherein R₄ is branched or unbranched C₁ to C₆ alkyl, aryl, aralkyl or NR₅R₆,

wherein R₅ and R₆ are each independently H, C₁ to C₈ alkyl, C₆ to C₁₂ aryl, C₆ to C₁₂ aralkyl or C₆ to C₁₂ cycloalkyl, each optionally substituted;

wherein R₃ is H or C₁ to C₆ alkyl;

wherein R₈ is H or t-butoxycarbonyl (Boc).

In one embodiment, the compound has the structure:

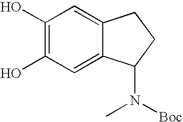

In another embodiment, the compound has the structure:

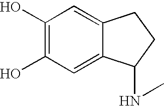

In still another embodiment, the compound has the structure:

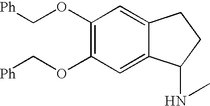

In an added embodiment, the compound has the structure:

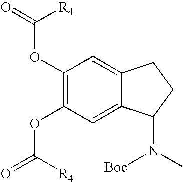

In yet another embodiment, R₄ is Ph.

In one embodiment, the compound has the structure:

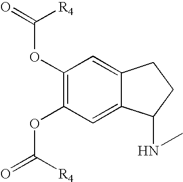

In a further embodiment, R₄ is Ph.

The subject invention additionally provides a pharmaceutical composition comprising a compound having the structure:

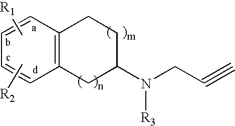

wherein R₁ is OC(O)R₉ and R₂ is H,

wherein R₉ is branched or unbranched C₁ to C₆ alkyl, aryl, or aralkyl, or

R₁ is OC(O)R₄ and R₂ is OC(O)R₄,

wherein R₄ is branched or unbranched C₁ to C₆ alkyl, aryl, aralkyl or NR₅R₆,

wherein R₅ and R₆ are each independently H, C₁ to C₈ alkyl, C₆ to C₁₂ aryl, C₆ to C₁₂ aralkyl or C₆ to C₁₂ cycloalkyl, each optionally substituted;

wherein R₃ is H or C₁ to C₆ alkyl;

wherein n is 0 or 1; and

wherein m is 1 or 2,

or a pharmaceutically acceptable salt thereof.

The subject invention further provides a pharmaceutical composition comprising a compound having the structure:

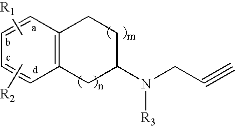

wherein R₁ is OH;

wherein R₂ is H or OC(O)R₄ when R₁ is attached to the “a” carbon or the “d” carbon, or

R₂ is OC(O)R₄ when R₁ is attached to the “b” carbon or the “c” carbon;

wherein R₄ is C₁ to C₆ branched or unbranched alkyl, aryl, aralkyl or NR₅R₆,

wherein R₅ and R₆are each independently H, C₁ to C₈ alkyl, C₆ to C₁₂ aryl, C₆ to C₁₂ aralkyl or C₆ to C₁₂ cycloalkyl, each optionally substituted;

wherein n is 0 or 1, and m is 1 or 2; and

wherein R₃ is H or Me when n is 1 and m is 1, or R₃ is H or C₁ to C₆ alkyl when n is 0 or m is 2,

or a pharmaceutically acceptable salt thereof.

The subject invention also provides a pharmaceutical composition comprising a compound having the structure:

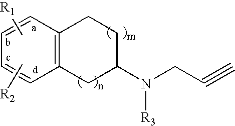

wherein the compound is an optically pure enantiomer;

wherein R₁ is OH;

wherein R₂ is H;

wherein R₃ is H or C₁ to C₆ alkyl;

wherein n is 0 or 1; and

wherein m is 1 or 2,

or a pharmaceutically acceptable salt thereof.

The subject invention also provides a method of treating a subject afflicted with a neurological disease comprising administering to the subject a compound having the structure:

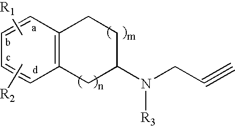

wherein R₁ is OH or OC(O)R₄;

wherein R₂ is H, OH or OC(O)R₄,

wherein R₄ is branched or unbranched C₁ to C₆ alkyl, aryl, aralkyl or NR₅R₆,

wherein R₅ and R₆ are each independently H, C₁ to C₈ alkyl, C₆ to C₁₂ aryl, C₆ to C₁₂ aralkyl or C₆ to C₁₂ cycloalkyl, each optionally substituted;

wherein R₃ is H or C₁ to C₆ alkyl;

wherein n is 0 or 1; and

wherein m is 1 or 2,

or a pharmaceutically acceptable salt thereof, or a prodrug which becomes the compound in the subject, so as to thereby treat the neurological disease in the subject.

Additionally, the subject invention provides a method of treating a subject afflicted with a neurological disease comprising administering to the subject a compound having the structure:

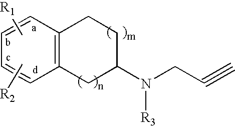

wherein R₁ is OH or OC(O)R₉ and R₂ is H or OC(O)R₄, or both R₁ and R₂ are OC(O)R₄,

wherein R₉ is branched or unbranched C₁ to C₆ alkyl, aryl, or aralkyl;

wherein R₄ is branched or unbranched C₁ to C₆ alkyl, aryl, aralkyl or NR₅R₆,

wherein R₅ and R₆ are each independently H, C₁ to C₈ alkyl, C₆ to C₁₂ aryl, C₆ to C₁₂ aralkyl or C₆ to C₁₂ cycloalkyl, each optionally substituted;

wherein R₃ is H or C₁ to C₆ alkyl;

wherein n is 0 or 1; and

wherein m is 1 or 2,

or a pharmaceutically acceptable salt thereof, or a prodrug which becomes the compound in the subject, so as to thereby treat the neurological disease in the subject.

In one embodiment of the method, the compound has the structure:

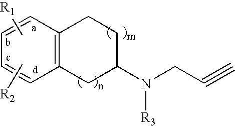

wherein R₁ is OC(O)R₉ and R₂ is H,

wherein R₉ is branched or unbranched C₁ to C₆ alkyl, aryl, or aralkyl, or

R₁ is OC(O)R₄ and R₂ is OC(O)R₄,

wherein R₄ is branched or unbranched C₁ to C₆ alkyl, aryl, aralkyl or NR₅R₆,

wherein R₅ and R₆are each independently H, C₁ to C₈ alkyl, C₆ to C₁₂ aryl, C₆ to C₁₂ aralkyl or C₆ to C₁₂ cycloalkyl, each optionally substituted;

wherein R₃ is H or C₁ to C₆ alkyl;

wherein n is 0 or 1; and

wherein m is 1 or 2.

In another embodiment of the method, the compound has the structure:

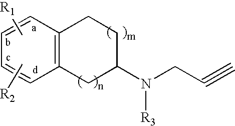

wherein R₁ is OH;

wherein R₂ is H or OC(O)R₄ when R₁ is attached to the “a” carbon or the “d” carbon, or

R₂ is OC(O)R₄ when R₁ is attached to the “b” carbon or the “c” carbon;

wherein R₄ is C₁ to C₆ branched or unbranched alkyl, aryl, aralkyl or NR₅R₆,

wherein R₅ and R₆ are each independently H, C₁ to C₈ alkyl, C₆ to C₁₂ aryl, C₆ to C₁₂ aralkyl or C₆ to C₁₂ cycloalkyl, each optionally substituted;

wherein R₃ is H or C₁ to C₆ alkyl;

wherein n is 0 or 1; and

wherein m is 1 or 2.

In a further embodiment of the method, the compound has the structure:

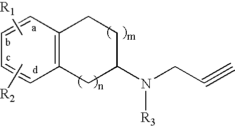

wherein the compound is an optically pure enantiomer;

wherein R₁ is OH;

wherein R₂ is H;

wherein R₃ is H or C₁ to C₆ alkyl;

wherein n is 0 or 1; and

wherein m is 1 or 2.

In one embodiment, the subject is human.

In a further embodiment, the administration comprises oral, parenteral, intravenous, transdermal, or rectal administration.

In one embodiment, the effective amount is from about 0.01 mg per day to about 100.0 mg per day.

In yet another embodiment, the effective amount is from about 0.01 mg per day to about 50.0 mg per day.

In still another embodiment, the effective amount is from about 0.1 mg per day to about 100.0 mg per day.

In an added embodiment, the effective amount is from about 0.1 mg per day to about 10.0 mg per day.

In yet another embodiment, the effective amount is from about 0.01 mg to about 100.0 mg.

In one embodiment, the effective amount is from about 0.01 mg to about 50.0 mg.

In a further embodiment, the effective amount is from about 0.1 mg to about 100.0 mg.

In another embodiment, the effective amount is from about 0.1 mg to about 10.0 mg.

In an additional embodiment, the neurological disease is Parkinson's disease, Alzheimer's disease, depression, epilepsy, narcolepsy, amyotrophic lateral sclerosis (ALS), memory disorders, panic, post-traumatic stress disorder (PTSD), sexual dysfunction, attention deficit and hyperactivity syndrome (ADHD), attention deficit disorder, or Tourette's syndrome. The disease may also be neuropathy, hyperactive syndrome, neurotrauma, stroke, Parkinson's disease, Huntington's disease, and other dementia such as senile dementia, dementia of the vascular dementia or Lewy body dementia.

In still another embodiment, the neurological disease is depression.

In still another embodiment, the compound has the structure:

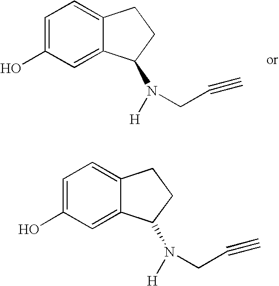

The subject invention further provides a process for preparing a compound having the structure:

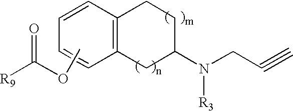

wherein n is 0 or 1, and m is 1 or 2;

wherein R₃ is H or C₁ to C₆ alkyl; and

wherein R₉ is branched or unbranched C₁ to C₆ alkyl, aryl, or aralkyl;

comprising the step of reacting

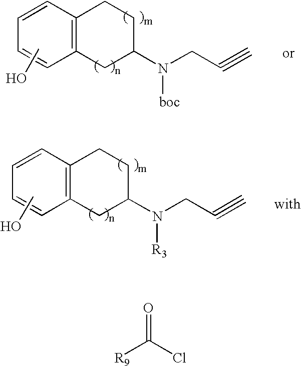

in the presence of an acid or 4-dimethylaminopyridine (DMAP) to form the compound.

The subject invention also provides a process for preparing a compound having the structure:

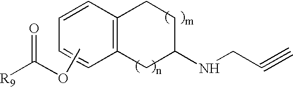

wherein R₉ is branched or unbranched C₁ to C₆ alkyl, aryl, or aralkyl;

which process comprises:

(a) reacting a compound having the structure:

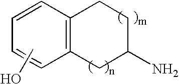

with a compound having the structure:

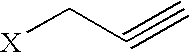

wherein X is a leaving group,

to produce a compound having the structure:

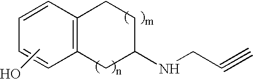

(b) reacting the compound formed in step (a) with a compound having the structure:

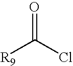

in the presence of trifluoroacetic acid (TFA) and an aprotic solvent to produce a compound having the structure:

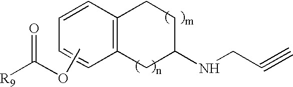

In one embodiment, the leaving group in step (a) is selected from the group consisting of a halogen and benzene sulfonate and the aprotic solvent in step (b) is CHCl₃.

The subject invention further provides a process for preparing a compound having the structure:

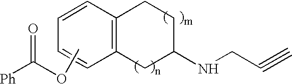

which comprises:

(a) reacting a compound having the structure:

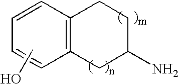

with a compound having the structure:

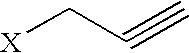

wherein X is a leaving group,

to produce a compound having the structure:

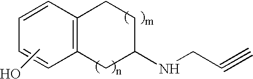

(b) N-protecting the compound formed in step (a) with tert-butoxycarbonyl (Boc) to produce a compound having the structure:

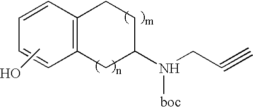

(c) reacting the compound formed in step (b) with a compound having the structure:

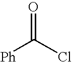

in the presence of 4-dimethylaminopyridine (DMAP) to produce a compound having the structure:

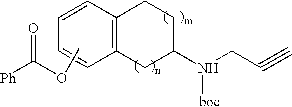

(d) deprotecting the compound formed in step (c) with HCl to produce a compound having the structure:

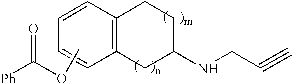

In one embodiment, the leaving group in step (a) is selected from the group consisting of a halogen and benzene sulfonate and the aprotic solvent in step (b) is CHCl₃.

The subject invention additionally provides a process for preparing a compound having the structure:

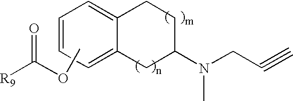

wherein R₉ is branched or unbranched C₁ to C₆ alkyl, aryl, or aralkyl;

which process comprises:

(a) reacting a compound having the structure:

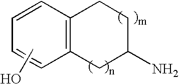

with a compound having the structure:

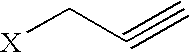

wherein X is a leaving group,

to produce a compound having the structure:

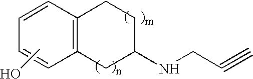

(b) reacting the compound formed in step (a) with NaCNBH₃ and paraformaldehyde to produce a compound having the structure:

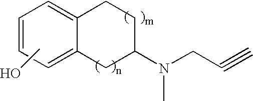

(c) reacting the compound formed in step (b) with a compound having the structure:

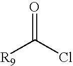

in the presence of trifluoroacetic acid (TFA) and an aprotic solvent to form a compound having the structure:

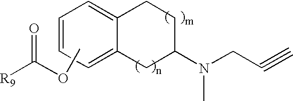

In one embodiment, the leaving group in step (a) is selected from the group consisting of a halogen and benzene sulfonate and the aprotic solvent in step (c) is CHCl₃.

The subject invention provides another process for preparing a compound having the structure:

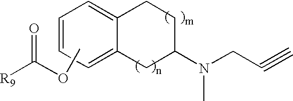

wherein R₉ is branched or unbranched C₁ to C₆ alkyl, aryl, or aralkyl;

which process comprises:

(a) reacting a compound having the structure:

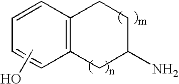

with ethyl formate to produce a compound having the structure:

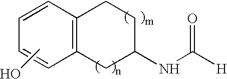

(b) reacting the compound formed in step (a) with lithium aluminum hydride to produce a compound having the structure:

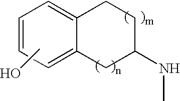

(c) reacting the compound formed in step (b) with a compound having the structure:

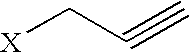

wherein X is a leaving group,

to form a compound having the structure:

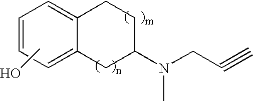

(d) reacting the compound formed in step (c) with a compound having the structure:

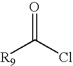

in the presence of trifluoroacetic acid (TFA) and an aprotic solvent to form a compound having the structure:

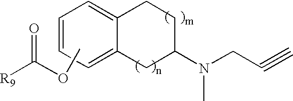

In one embodiment, the aprotic solvent in step (c) is CHCl₃.

The subject invention provides yet another process for preparing a compound having the structure:

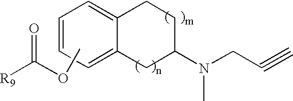

wherein R₉ is branched or unbranched C₁ to C₆ alkyl, aryl, or aralkyl;

which process comprises:

(a) reacting a compound having the structure:

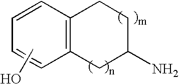

with NaCNBH₃/paraformaldehyde to produce a compound having the structure:

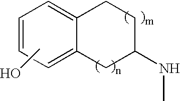

(b) reacting the compound formed in step (a) with a compound having the structure:

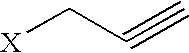

wherein X is a leaving group,

to form a compound having the structure:

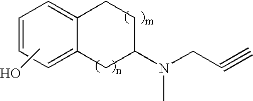

(c) reacting the compound formed in step (b) with a compound having the structure:

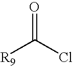

in the presence of trifluoroacetic acid (TFA) and an aprotic solvent to form a compound having the structure:

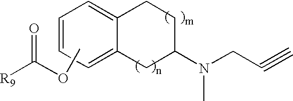

In one embodiment, the aprotic solvent in step (d) is CHCl₃.

Additionally, the subject invention provides a process for preparing a compound having the structure:

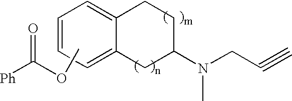

which comprises:

(a) reacting a compound having the structure:

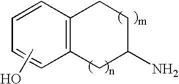

with a compound having the structure:

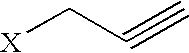

wherein X is a leaving group,

to produce a compound having the structure:

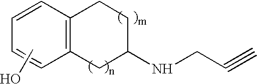

(b) reacting the compound formed in step (a) with NaCNBH₃ and paraformaldehyde to produce a compound having the structure:

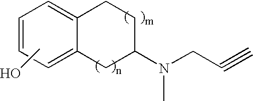

(c) reacting the compound formed in step (b) with a compound having the structure:

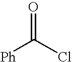

in the presence of 4-dimethylaminopyridine (DMAP) and an aprotic solvent to form a compound having the structure:

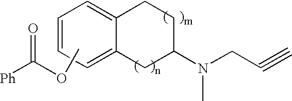

In one embodiment, the leaving group in step (a) is selected from the group consisting of a halogen and benzene sulfonate and the aprotic solvent in step (c) is CHCl₃.

The subject invention provides another process for preparing a compound having the structure:

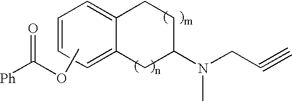

which comprises:

(a) reacting a compound having the structure:

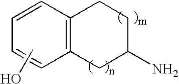

with ethyl formate to produce a compound having the structure:

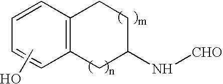

(b) reacting the compound formed in step (a) with lithium aluminum hydride to produce a compound having the structure:

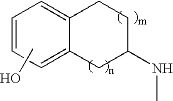

(c) reacting the compound formed in step (b) with a compound having the structure:

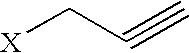

wherein X is a leaving group,

to form a compound having the structure:

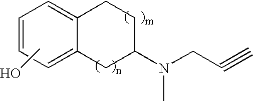

(d) reacting the compound formed in step (c) with a compound having the structure:

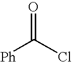

in the presence of 4-dimethylaminopyridine (DMAP) and an aprotic solvent to form a compound having the structure:

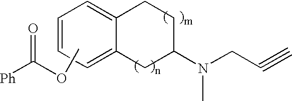

In one embodiment, the aprotic solvent in step (c) is CHCl₃.

The subject invention provides yet another process for preparing a compound having the structure:

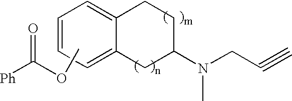

which comprises:

(a) reacting a compound having the structure:

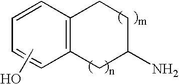

with NaCNBH₃/paraformaldehyde to produce a compound having the structure:

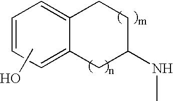

(b) reacting the compound formed in step (a) with a compound having the structure:

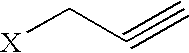

wherein X is a leaving group,

to form a compound having the structure:

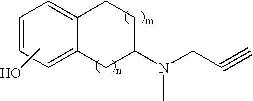

(c) reacting the compound formed in step (b) with a compound having the structure:

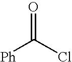

in the presence of 4-dimethylaminopyridine (DMAP) and an aprotic solvent to form a compound having the structure:

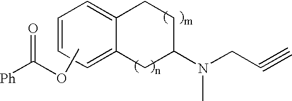

In one embodiment, the aprotic solvent in step (d) is CHCl₃.

The subject invention further provides a process for preparing a compound having the structure:

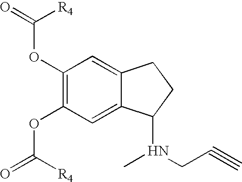

which comprises:

(a) reacting a compound having the structure:

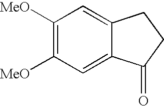

with AlCl₃ or BBr₃ in the presence of toluene to produce a compound having the structure:

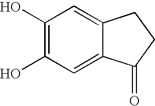

(b) reacting the product formed in step (a) with benzyl chloride and K₂CO₃ in the presence of dimethyl formamide (DMF) to produce a compound having the structure:

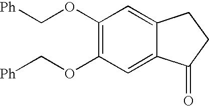

(c) reacting the product formed in step (b) with MeNH₂.HCl, NaCNBH₃ in tetrahydrofuran (THF)/MeOH to produce a compound having the structure:

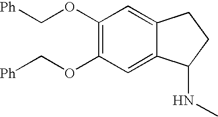

(d) reacting the product formed in step (c) with H₂, Pd/C and MeOH to produce a compound having the structure:

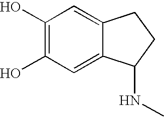

(e) reacting the product formed in step (d) with Boc₂O, dioxane/H₂O and NaHCO₃ to produce a compound having the structure:

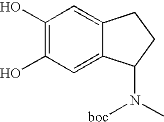

(f) reacting the product formed in step (e) with R₄COCl, Et₃N in CH₂Cl₂ in the presence of 4-dimethylaminopyridine (DMAP) to produce a compound having the structure:

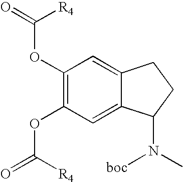

(g) reacting the product formed in step (f) with HCl/dioxane to produce a compound having the structure:

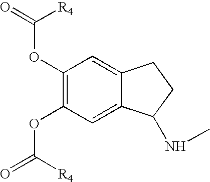

(h) reacting the product formed in step (g) with propargyl bromide, K₂CO₃ in CH₃CN and then with HCl/ether and MeOH to produce a compound having the structure:

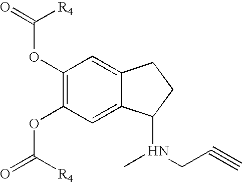

Also, the subject invention provides a process for preparing a compound having the structure:

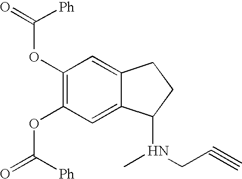

which comprises:

(a) reacting a compound having the structure:

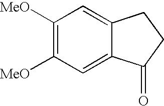

with AlCl₃ or BBr₃ in the presence of toluene to produce a compound having the structure:

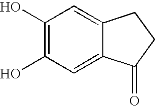

(b) reacting the product formed in step (a) with benzyl chloride and K₂CO₃ in the presence of dimethyl formamide (DMF) to produce a compound having the structure:

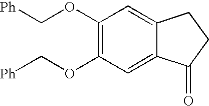

(c) reacting the product formed in step (b) with MeNH₂.HCl, NaCNBH₃ in tetrahydrofuran (THF)/MeOH to produce a compound having the structure:

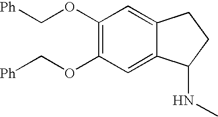

(d) reacting the product formed in step (c) with H₂, Pd/C and MeOH to produce a compound having the structure:

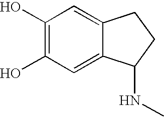

(e) reacting the product formed in step (d) with Boc₂O, dioxane/H₂O and NaHCO₃ to produce a compound having the structure:

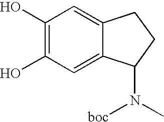

(f) reacting the product formed in step (e) with PhCOCl, Et₃N in CH₂Cl₂ in the presence of 4-dimethylaminopyridine (DMAP) to produce a compound having the structure:

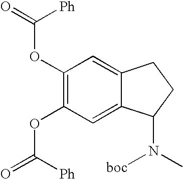

(g) reacting the product formed in step (f) with HCl/dioxane to produce a compound having the structure:

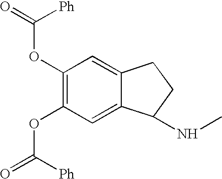

(h) reacting the product formed in step (g) with propargyl bromide, K₂CO₃ in CH₃CN and then with HCl/ether and MeOH to produce a compound having the structure:

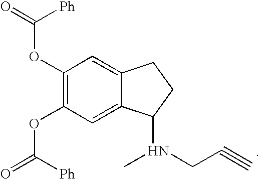

The subject invention further provides the use of a compound or a prodrug of a compound which becomes the compound having the structure:

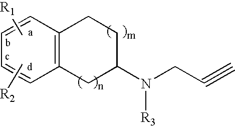

wherein R₁ is OH or OC(O)R₄;

wherein R₂ is H, OH or OC(O)R₄,

wherein R₄ is branched or unbranched C₁ to C₆ alkyl, aryl, aralkyl or NR₅R₆,

wherein R₅ and R₆ are each independently H, C₁ to C₈ alkyl, C₆ to C₁₂ aryl, C₆ to C₁₂ aralkyl or C₆ to C₁₂ cycloalkyl, each optionally substituted;

wherein R₃ is H or C₁ to C₆ alkyl;

wherein n is 0 or 1; and

wherein m is 1 or 2,

or a pharmaceutically acceptable salt thereof, for the manufacture of a medicament for treating a subject afflicted with a neurological disease, wherein the compound is to be periodically administered to the subject in a therapeutically effective dose.

The subject invention also provides the use of a compound or a prodrug of a compound which becomes the compound having the structure:

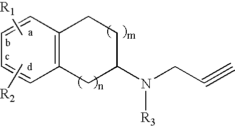

wherein R₁ is OH or OC(O)R₉, and

wherein R₉ is branched or unbranched C₁ to C₆ alkyl, aryl, or aralkyl;

R₂ is H or OC(O)R₄, or both R₁ and R₂ are OC(O)R₄,

wherein R₄ is branched or unbranched C₁ to C₆ alkyl, aryl, aralkyl or NR₅R₆,

wherein R₅ and R₆ are each independently H, C₁ to C₈ alkyl, C₆ to C₁₂ aryl, C₆ to C₁₂ aralkyl or C₆ to C₁₂ cycloalkyl, each optionally substituted;

wherein R₃ is H or C₁ to C₆ alkyl;

wherein n is 0 or 1; and

wherein m is 1 or 2,

or a pharmaceutically acceptable salt thereof, for the manufacture of a medicament for treating neurological disease in a subject, wherein the compound is to be periodically administered to the subject in a therapeutically effective dose.

In one embodiment of the use, the compound has the structure:

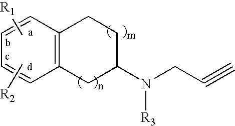

wherein R₁ is OC(O)R₉ and R₂ is H,

wherein R₉ is branched or unbranched C₁ to C₆ alkyl, aryl, or aralkyl, or

R₁ is OC(O)R₄ and R₂ is OC(O)R₄,

wherein R₄ is branched or unbranched C₁ to C₆ alkyl, aryl, aralkyl or NR₅R₆,

wherein R₅ and R₆ are each independently H, C₁ to C₈ alkyl, C₆ to C₁₂ aryl, C₆ to C₁₂ aralkyl or C₆ to C₁₂ cycloalkyl, each optionally substituted;

wherein R₃ is H or C₁ to C₆ alkyl;

wherein n is 0 or 1; and

wherein m is 1 or 2.

In another embodiment of the use, the compound has the structure:

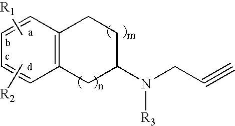

wherein R₁ is OH;

wherein R₂ is H or OC(O)R₄ when R₁ is attached to the “a” carbon or the “d” carbon, or

R₂ is OC(O)R₄ when R₁ is attached to the “b” carbon or the “c” carbon;

wherein R₄ is C₁ to C₆ branched or unbranched alkyl, aryl, aralkyl or NR₅R₆,

wherein R₅ and R₆ are each independently H, C₁ to C₈ alkyl, C₆ to C₁₂ aryl, C₆ to C₁₂ aralkyl or C₆ to C₁₂ cycloalkyl, each optionally substituted;

wherein R₃ is H or C₁ to C₆ alkyl;

wherein n is 0 or 1; and

wherein m is 1 or 2.

In an additional embodiment of the use, the compound has the structure:

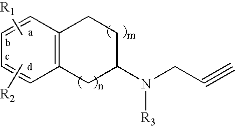

wherein the compound is an optically pure enantiomer;

wherein R₁ is OH;

wherein R₂ is H;

wherein R₃ is H or C₁ to C₆ alkyl;

wherein n is 0 or 1; and

wherein m is 1 or 2.

In a further embodiment of the use, the subject is human.

In yet another embodiment of the use, the medicament is formulated for oral, parenteral, intravenous, transdermal, or rectal administration.

In an embodiment of the use, the therapeutically effective amount is from about 0.01 mg per day to about 50.0 mg per day.

In an added embodiment of the use, the therapeutically effective amount is from about 0.1 mg per day to about 100.0 mg per day.

In still another embodiment of the use, the therapeutically effective amount is from about 0.1 mg per day to about 10.0 mg per day.

In an embodiment of the use, the neurological disease is Parkinson's disease, Alzheimer's disease, depression, epilepsy, narcolepsy, amyotrophic lateral sclerosis (ALS), memory disorders, panic, post-traumatic stress disorder (PTSD), sexual dysfunction, attention deficit and hyperactivity syndrome (ADHD), attention deficit disorder, or Tourette's syndrome.

In a further embodiment of the use, the neurological disease is depression. In one embodiment, the compound has the structure:

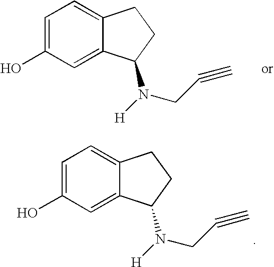

The subject invention thus discloses various derivatives and isomers of hydroxylated propargylamino indan and tetralin which have surprisingly varied potency and selectivity for MAO inhibition. The subject invention also provides modifications of the hydroxy compounds which have surprisingly varied MAO inhibitory properties depending upon the substitution pattern, however, the hydroxy compound is always a more potent inhibitor than the modified version. Thus, the modified version may be considered a prodrug of the more active hydroxy compound into which it will be metabolized in vivo.

In one embodiment of the invention, the prodrug compound is a carboxylic acid ester of the hydroxy compound. In another embodiment, the parent is a carbamate derivative of the hydroxy compound.

As discussed above, carbamate propargylamino indans and tetralins have been reported in PCT International Application No. PCT/US97/24155 as both MAO inhibitors and AchE inhibitors. However, it is a further embodiment of this invention that such a prodrug compound will not be a potent inhibitor of AchE (IC₅₀>500 micromolar), and the IC₅₀ for MAO-A inhibition of the corresponding hydroxy metabolite be at least 100 times more potent than the prodrug.

In one embodiment, the compounds are dihydroxy derivatives of propargylamino indan or tetralin. These derivatives are expected to be antioxidants, as well as MAO inhibitors. In another embodiment, the subject invention provides ester prodrugs.

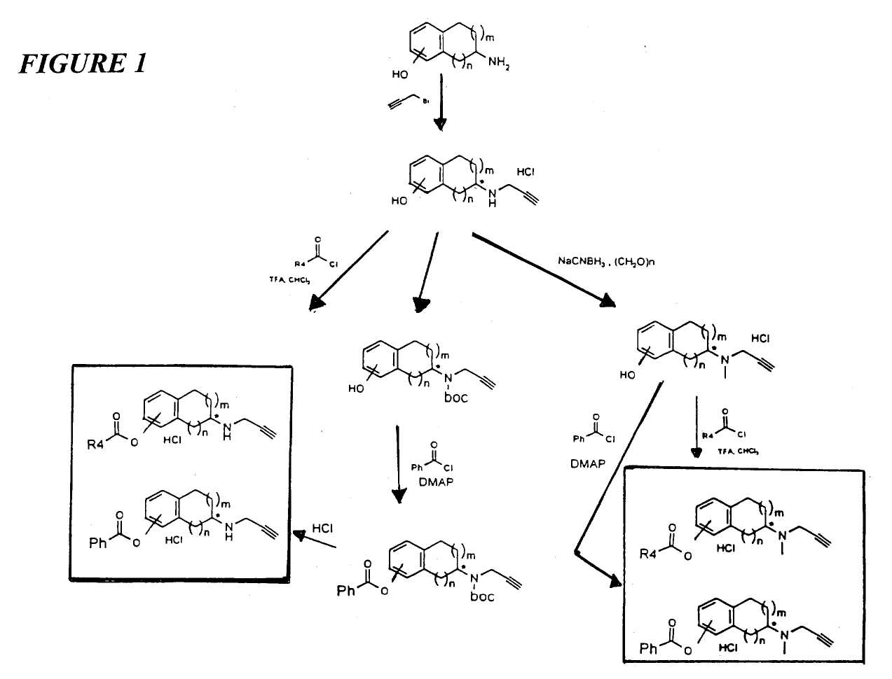

Thus, the subject invention provides esters or carbamates of propargylamino indanols, propargylamino indandiols, propargylamino tetralinols or propargylamino tetralindiols, and may be prepared by methods of esterification or carbamoylation of hydroxy compounds. Ester derivatives (FIG. 1) when R₂ equals hydrogen were prepared by reacting the propargylamino indanols with acyl chlorides in the presence of a strong organic acid such as trifluoroacetic acid or an acylation catalyst such as 4-dimethylaminopyridine (DMAP), with or without an inert organic solvent such as chloroform. Compounds when R₃ equals hydrogen were prepared either by direct acylation as described above, or by first N-protecting the amine moiety, e.g., by a tert-butoxycarbonyl (Boc) group, followed by acylation as above, and finally removing the protecting group. The preparation of compounds of the subject invention which are carbamates is described in PCT/US97/24155.

Propargylamino indanols may be prepared by reacting amino indanols with propargyl bromide in a polar organic solvent such as N,N-dimethylacetamide or acetonitrile in the presence of a base such as potassium carbonate. N-Methyl, N-propargylamino indanols may be prepared by reductive alkylation of propargylamino indanols by methods known to those skilled in the art, e.g., with NaCNBH₃ and paraformaldehyde. Alternatively, N-methyl,N-propargylamino indanols were prepared by first methylating amino indanols either by NaCNBH₃/paraformaldehyde or by ethyl formate followed by LiAlH₄ reduction, and then reacting the N-methylamino indanols thus obtained with propargyl bromide as described above.

The N-propargyl derivatives of, inter alia, 3-amino-indan-4-ol, 1-amino-indan-4-ol, 3-amino-indan-5-ol and 7-amino-5,6,7,8-tetrahydro-naphthalen-2-ol were prepared.

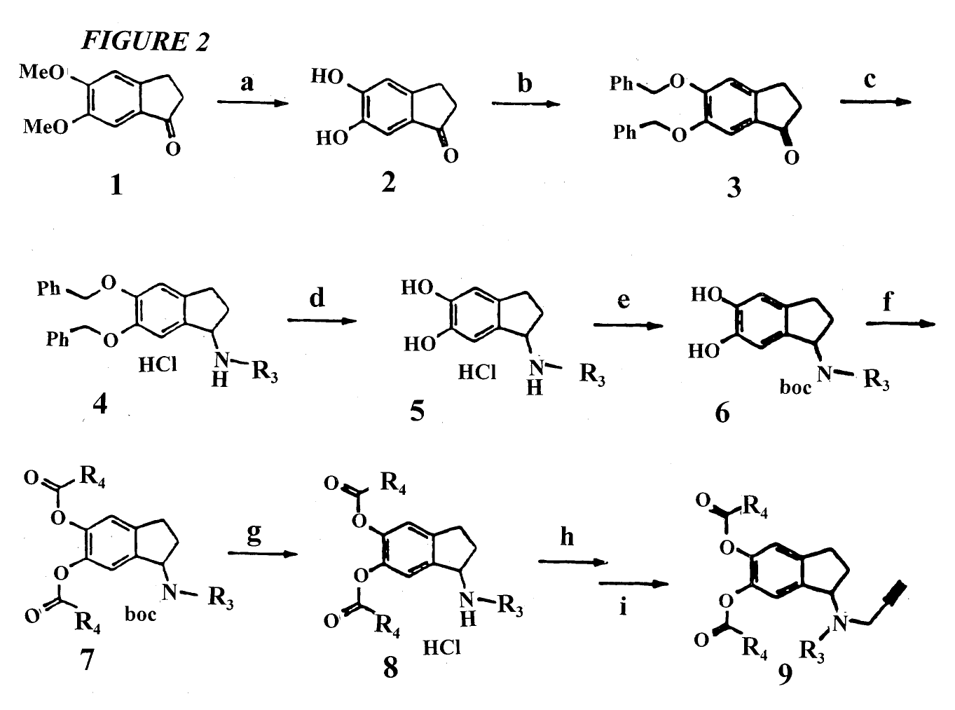

Compounds of the subject invention with both R₁ and R₂ equal to OCOR₄ (see FIG. 2, compound numbered 9) were prepared by propargylation of 5,6-di-O-benzoyl-1-methylamino-1-indan (FIG. 2, compound numbered 8), as described above. 5,6-Di-O-benzoyl-1-methylamino-1-indan (FIG. 2, compound numbered 8) was prepared from 5,6-bis-benzyloxy-1-indanone 3 as follows:

1) reductive amination of the compound numbered 3 in FIG. 2 as described above gave 5,6-bis-benzyloxy-1-indanyl)methylamine (FIG. 2, compound numbered 4);

2) the compound numbered 4 in FIG. 2 was debenzylated by catalytic hydrogenation and protected by the Boc group to give N-Boc-1-methylamino-indan-5,6-diol (FIG. 2, compound numbered 6); and

3) Compound 6 in FIG. 2 was esterified as described above and the protecting group removed as previously described to give 5,6-di-O-benzoyl-1-methylamino-1-indan (FIG. 2, compound numbered 8).

The diester tetralin derivative numbered 12 (FIG. 3) was prepared by esterification of the dihydroxy tetralin numbered 11 (FIG. 3).

Chemical Data

| cmpd | | | | R₁ | | | | | | yield |
|---|---|---|---|---|---|---|---|---|---|---|
| # | ster | R₂ | R₁ | pos | R₃ | n | m | mp | formula | (%) |
| 100* | S | H | OH | 6 | H | 0 | 1 | 175-7 | C₁₃H₁₇NO₄S | 45 |
| 101* | R | H | OH | 6 | H | 0 | 1 | 173-5 | C₁₃H₁₇NO₄S | 42 |
| 102 | S | H | OCOMe | 6 | H | 0 | 1 | 138-40 | C₁₄H₁₆CINO₂ | 46 |
| 103 | R | H | OCOMe | 6 | H | 0 | 1 | 156-8 | C₁₄H₁₆CINO₂ | 77 |
| 104 | S | H | OCOtBu | 6 | H | 0 | 1 | 126-8 | C₁₇H₂₂CINO₂ | 67 |
| 105 | R | H | OCOtBu | 6 | H | 0 | 1 | 128-30 | C₁₇H₂₂CINO₂ | 46 |
| 106 | S | H | OCOnBu | 6 | H | 0 | 1 | 149-50 | C₁₇H₂₂CINO₂ | 37 |
| 107 | R | H | OCOnBu | 6 | H | 0 | 1 | 155-7 | C₁₇H₂₂CINO₂ | 85 |
| 108 | S | H | OCOCH₂Ph | 6 | H | 0 | 1 | 144-5 | C₂₀H₂₀CINO₂ | 22 |
| 109 | R | H | OCOCH₂Ph | 6 | H | 0 | 1 | 145-7 | C₂₀H₂₀CINO₂ | 52 |
| 110 | S | H | OCOPh | 6 | H | 0 | 1 | 202-4 | C₁₉H₁₈CINO₂ | 18 |
| 111 | R | H | OCOPh | 6 | H | 0 | 1 | 210-11 | C₁₉H₁₈CINO₂ | 61 |
| 112 | rac | H | OH | 6 | Me | 0 | 1 | 210-11 | C₁₃H₁₆CINO | 70 |
| 113 | S | H | OH | 6 | Me | 0 | 1 | 82-4 | C₁₃H₁₆CINO | 72 |
| 114 | R | H | OH | 6 | Me | 0 | 1 | 71-2 | C₁₃H₁₆CINO | 78 |
| 115 | S | H | OCOMe | 6 | Me | 0 | 1 | 168-70 | C₁₅H₁₈CINO₂ | 95 |
| 116 | R | H | OCOMe | 6 | Me | 0 | 1 | 168-70 | C₁₅H₁₈CINO₂ | 93 |
| 117 | rac | H | OH | 4 | Me | 0 | 1 | 160-62 | C₁₃H₁₆NCIO | 89 |
| 118 | rac | H | OH | 7 | Me | 0 | 1 | 83-5 | C₁₃H₁₆NCIO | 53 |
| 119 | rac | H | OCOMe | 4 | Me | 0 | 1 | 148-50 | C₁₅H₁₈CINO₂ | 72 |
| 120 | rac | H | OCOPh | 4 | Me | 0 | 1 | 176-8 | C₂₀H₂₀CINO₂ | 59 |
| 121 | rac | H | OCOPh(OMe)₂ | 4 | Me | 0 | 1 | 183-5 | C₂₂H₂₄CINO₄ | 39 |
| 122 | rac | H | OCOPh | 7 | Me | 0 | 1 | 185-7 | C₂₀H₂₀CINO₂ | 45 |
| 123 | rac | H | OH | 7 | Me | 1 | 1 | 220-1 | C₁₄H₁₈NCIO | 66 |
| 124 | rac | H | OCOPh | 7 | Me | 1 | 1 | 104-6 | C₂₁H₂₂CINO₂ | 71 |
| 125 | S | H | OCOnBu | 6 | Me | 0 | 1 | 78-80 | C₁₈H₂₄CINO₂ | 73 |
| 126 | R | H | OCOnBu | 6 | Me | 0 | 1 | 96-8 | C₁₈H₂₄CINO₂ | 72 |
| 127 | S | H | OCOPh | 6 | Me | 0 | 1 | 73-5 | C₂₀H₂₀CINO₂ | 52 |
| 128 | R | H | OCOPh | 6 | Me | 0 | 1 | 82-4 | C₂₀H₂₀CINO₂ | 56 |
| 129 | S | H | OCOtBu | 6 | Me | 0 | 1 | 153-5 | C₁₈H₂₄CINO₂ | 73 |
| 130 | R | H | OCOtBu | 6 | Me | 0 | 1 | 155-7 | C₁₈H₂₄CINO₂ | 78 |
| 131 | S | H | OCOPh(Me) | 6 | Me | 0 | 1 | ** | C₂₁H₂₂CINO₂ | 51 |
| 132 | R | H | OCOPh(Me) | 6 | Me | 0 | 1 | 82-4 | C₂₁H₂₂CINO₂ | 46 |
| 133 | S | H | OCOPh(OMe)₂ | 6 | Me | 0 | 1 | 118-20 | C₂₂H₂₄CINO₂ | 58 |
| 134 | R | H | OCOPh(OMe)₂ | 6 | Me | 0 | 1 | 73-5 | C₂₂H₂₄CINO₂ | 68 |
| 135 | rac | H | OH | 7 | H | 0 | 1 | 166-8 | C₁₂H₁₄CINO | 35 |
| 136 | rac | H | OH | 4 | H | 0 | 1 | 196-8 | C₁₂H₁₄CINO | 66 |
| 137 | rac | OCOPh | OCOPh | 6 | Me | 0 | 1 | 114-5 | C₂₇H₂₄CINO₄ | 59 |
| | | (5-pos) | | | | | | | | |
| 138 | rac | OCOPh | OCOPh | 7 | Me | 1 | 1 | 180-2 | C₂₈H₂₆CINO₄ | 58 |
| | | (6-pos) | | | | | | | | |

ster = stereochemistry
pos = position
*mesylesylate salts
**wide range, hygroscopic

TABLE 2
¹H-NMR Data (R₁ = R₃ = H) (300 MHz, dimethyl sulfoxide
(DMSO) -d₆)

| Cmpd | | indan | | | Pg | | | |
|---|---|---|---|---|---|---|---|---|
| # | Ph | C3-H | C2-H | C1-H | CH2 | CH | R₄ | NH₂ |
| | 7.52 (d) | | | | | | | |
| 102 | 7.35 (d) | 4.79 (m) | 2.43 (m) | 2.83 (m) | 3.88 (m) | 3.71 (m) | 2.27 (Me,s) | 10.2 (br s) |
| 103 | 7.10 (dd) | | 2.28 (m) | 3.12 (m) | | | | |
| | 7.48 (d) | | | | | | | |
| 104 | 7.36 (d) | 4.79 (m) | 2.45 (m) | 2.85 (m) | 3.90 (m) | 3.72 (m) | 1.30 (tBu,s) | 10.15 |
| 105 | 7.07 (dd) | | 2.27 (m) | 3.12 (m) | | | | (br s) |
| | 7.48 (d) | | | | | | | |
| 108 | 7.36 (d) | 4.80 (m) | 2.45 (m) | 2.85 (m) | 3.91 (m) | 3.72 (m) | 7.38 (m,1H) | 10.2 |
| 109 | 7.07 (dd) | | 2.28 (m) | 3.13 (m) | | | 7.33 (m,4H) | (br s) |
| | | | | | | | 3.99 (CH2,s) | |
| | 7.48 (d) | | | | | | 2.57 (t,2H) | |
| 106 | 7.35 (d) | 4.79 (m) | 2.45 (m) | 2.85 (m) | 3.90 (m) | 3.71 (m) | 1.61 (m,2H) | 10.1 |
| 107 | 7.08 (dd) | | 2.26 (m) | 3.11 (m) | | | 1.38 (m,2H) | (brs) |
| | | | | | | | 0.9 1(t,3H) | |
| | 7.67 (d) | | | | | | | |
| 110 | 7.42 (d) | 4.83 (m) | 2.46 (m) | 2.86 (m) | 3.93 (m) | 3.72 (m) | 8.13 (d,2H) | 10.15 |
| 111 | 7.28 (dd) | | 2.30 (m) | 3.16 (m) | | | 7.76 (t,1H) | (d) |
| | | | | | | | 7.61 (t,2H) | |

TABLE 3
¹H-NMR Data (R₁ = H, R₃ = Me) (300 MHz,D₂O)

| Cmpd | | Indan | | | Propargyl | | | |
|---|---|---|---|---|---|---|---|---|
| # | Ph | C3-H | C2-H | C1-H | CH₂ | CH | R₄ | N-Me |
| | 7.50 (d) | | | | | | | |
| 116 | 7.35 (d) | 5.22 (m) | 2.46 (m) | 3.07 (m) | 4.05 (m) | 3.15 (m) | 2.37(Me,s) | 2.83 (s) |
| 115 | 7.25 (dd) | | 2.60 (m) | 3.17 (m) | | | | |
| | 7.50 (d) | | | | | | 2.69 (t,2H) | |
| 126 | 7.33 (d) | 5.23 (m) | 2.49 (m) | 3.07 (m) | 4.05 (m) | 3.17 (m) | 1.73 (m,2H) | 2.82 (s) |
| 125 | 7.22 (dd) | | 2.62 (m) | 3.17 (m) | | | 1.44 (m,2H) | |
| | | | | | | | 0.97 (t,3H) | |
| | 7.50 (d) | | | | | | 8.11 (dd,2H) | |
| 128 | 7.40 (d) | 5.17 (m) | 2.57 (m) | 3.06 (m) | 4.00 (m) | 3.15 (m) | 7.74 (dt,1H) | 2.81 (s) |
| 127 | 7.29 (dd) | | 2.47 (m) | 3.17 (m) | | | 7.57 (t,2H) | |
| | 7.49 (d) | | | | | | | |
| 129 | 7.28 (d) | 5.20 (m) | 2.60 (m) | 3.05 (m) | 4.03 (m) | 3.17 (m) | 1.37 (s,9H) | 2.81 (s) |
| 130 | 7.21 (dd) | | 2.45 (m) | 3.16 (m) | | | | |
| | 7.90 (d,1H) | | | | | | For Ar H's, | 2.72 (s) |
| 131 | 7.44 (t,1H) | 5.02 (m) | 2.50 (m) | 3.08 (m) | 3.93 (m) | 3.14 (m) | see | |
| 132 | 7.36 (m,2H) | | 2.40 (m) | 2.95 (m) | | | under Ph. | |
| | 7.21 (m,2H) | | | | | | 2.43 (s,Me) | |
| | 7.08 | | | | | | | |
| | (dd,1H) | | | | | | | |
| | | | | | | | For Ar H's, | 2.73 (s) |
| 133 | 7.5-7.1 | 5.05 | 2.50 (m) | 3.08( m) | 3.92 (m) | 3.16 (m) | see | |
| 134 | (m,4H) | (br d) | 2.41 (m) | 2.95 (m) | | | under Ph. | |
| | 6.74 (dd,2H) | | | | | | 3.84 (s,6H, | |
| | | | | | | | OMe) | |
| | 7.62 (t,1H) | 5.19 (m) | 2.46 (m) | 2.98 (m) | 4.09 (m) | 3.84 (s) | 8.13 (d,2H) | |
| 120* | 7.44 (t,1H) | | | 2.80 (m) | | | 7.77 (t,2H) | |
| | 7.35 (d,1H) | | | | | | 7.62 (t,1H) | |
| 119 | 7.50 (m,2H) | 5.29 (dd) | 2.60 (m) | 2.97 (m) | 4.05 (m) | 3.15 (m) | 2.39 (s,3H) | 2.81 (s) |
| | 7.26 (d,1H) | | 2.47 (m) | | | | | |
| 121 | 7.46 (t,2H) | 5.15 | 2.46 (m) | 2.93 (m) | 3.97 (m) | 3.16 (m) | 7.42 (t,1H) | 2.73 (s) |
| | 7.23 (dd, | (br d) | | | | | 6.75 (d,2H) | |
| | 1H) | | | | | | 3.83 (s,6H, | |
| | | | | | | | OMe) | |
| | 7.50 (t,1H) | 5.18 | 2.95 (m) | 3.50 (m) | 4.40- | 3.80(m) | 8.20 (dd,2H) | 2.68 (s) |
| 122* | 7.35 (d,1H) | (br s) | 2.36 (m) | | 3.90 (m) | | 7.78 (t,1H) | 2.56 (s) |
| | 7.25 (d,1H) | 4.96 | | | | | 7.61 (t,2H) | |
| | | (br s) | | | | | | |

*DM80-d6

TABLE 4
¹H-NMR Data (R₁ or R₂ = OH) (300 MHz, D₂O)

| | | | indian | | | propargyl | | |
|---|---|---|---|---|---|---|---|---|
| Cmpd # | structure | Ph | C3-H | C2-H | C1-H | CH₂ | CH | N-Me |
| 100 101 | 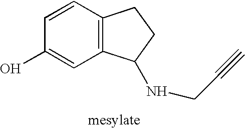 | 7.32 (d) 7.04 (d) 6.98 (dd) | 4.93 (dd) | 2,59 (m) 2.30 (m) | 3.06 (m) 2.96 (m) | 3.99 (m) | 3.06 (m) | |
| 135 | 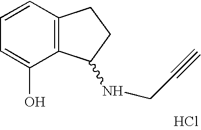 | 7.35 (t) 9.98 (d) 6.98 (d) | 5.07 (dd) | 2,57 (m) 2.30 (m) | 3.16 (m) 3.01 (m) | 4.01 (m) | 3.00 (m) | |
| 136 | 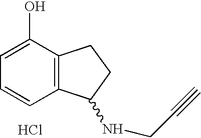 | 7.30 (d) 7.16 (d) 6.98 (d) | 4.99 (dd) | 2.60 (m) 2,32 (m) | 3.06 (m) 2.96 (m) | 4.02 (m) | 3.06 (m) | |
| 113 114 | 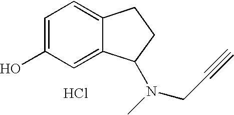 | 7.30 (d) 7.00 (m, 2H) | 5.1 (dd) | 2.52 (m) 2.41 (m) | 3.05 (m) 2.95 (m) | 4.00 (m) | 3.13 (m) | 2.78 (g) |
| 117 | 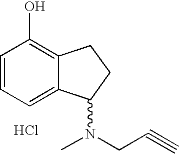 | 7.21 (t) 7.03 (d) 6.87 (d) | 5.09 (dd) | 2.40 (m) 2.30 (m) | 2.85 (m) | 3.90 (m) | 3.02 (g) | 2.65 (g) |
| 118 | 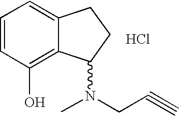 | 7.35 (t) 6.97 (d) 6.82 (d) | 5.30 (dd) | 2.50 (m) 2.39 (m) | 3.10 (m) 2.95 (m) | 4.06 (m) | 3.10 (g) | 2.79 (g) |

TABLE 5
MAO in vitro Data

| | | | In vitro IC₅₀(μM) | | |
|---|---|---|---|---|---|
| Cmpd # | | Structure | A | B | A/B |
| 101 | R | 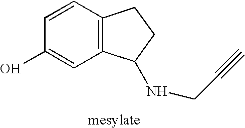 | 0.3 | 0.23 | 1.3 |
| 100 | S | | 500 | 300 | 1.66 |
| 112 | | 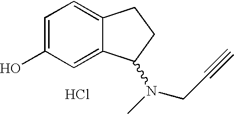 | 0.03 | 0.01 | 3.0 |
| 114 | R | | 0.01 | 0.03 | 0.33 |
| 113 | S | | 12 | 23 | 0.52 |
| 117 | | 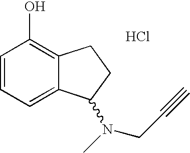 | 0.0083 | 0.07 | 0.1 |
| 118 | | 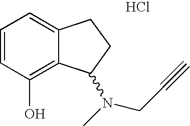 | 0.07 | 0.05 | 1.4 |
| 123 | | 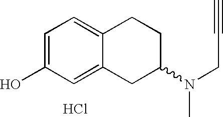 | 0.41 | 0.48 | 0.85 |
| 139 | | 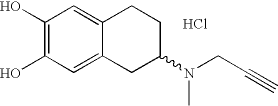 | 4.5 | 41 | 0.11 |
| 116 | | 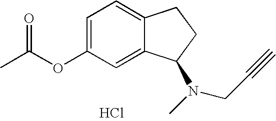 | 0.035 | 0.0056 | 6.25 |
| 126 | | 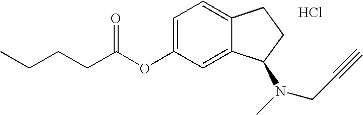 | 0.058 | 0.13 | 0.45 |
| 128 | R | 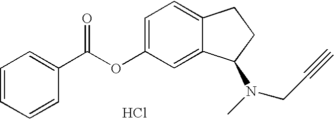 | 0.2 | 1.0 | 0.2 |
| 127 | S | | 3.8 | 5.6 | 0.68 |
| 130 | R | 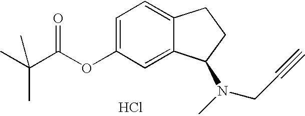 | 0.56 | 2.5 | 0.22 |
| 129 | S | | 2.5 | 17 | 0.15 |
| 132 | | 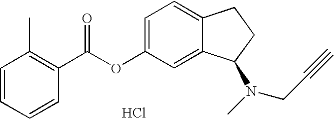 | 1.1 | 9.9 | 0.11 |
| 134 | | 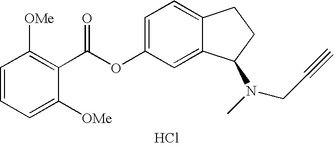 | 1.2 | 5.9 | 0.20 |
| 137 | | 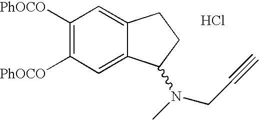 | 1.4 | 21.0 | 0.07 |
| 120 | | 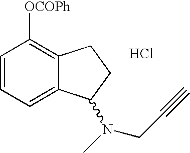 | 0.13 | 0.91 | 0.14 |
| 119 | | 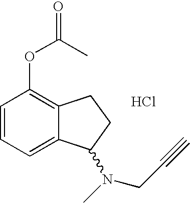 | 0.018 | 0.11 | 0.16 |
| 121 | | 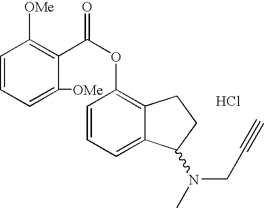 | 0.34 | 3 | 0.11 |
| 124 | | 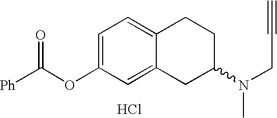 | 1.2 | 1.2 | 1 |
| 138 | | 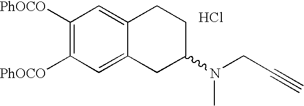 | 1.2 | 0.4 | 3 |
| 140 | | 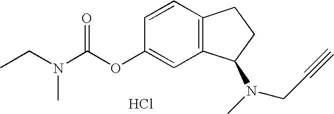 | 4.3 | 12 | 0.36 |
| 141 | | 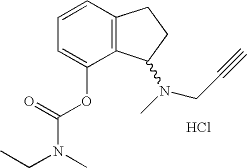 | 65 | 100 | 0.65 |
| 142 | | 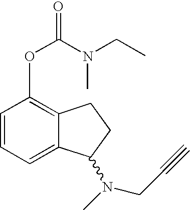 | 0.027 | 4 | 0.007 |
| 143 | | 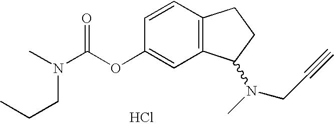 | 5.6 | 9.2 | 0.61 |
| 144 | | 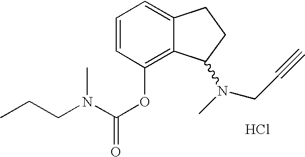 | 71 | 63 | 1.1 |
| 145 | | 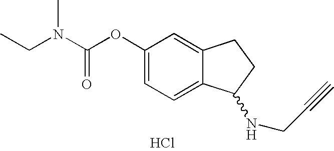 | ??? | ??? | |
| 146 | | 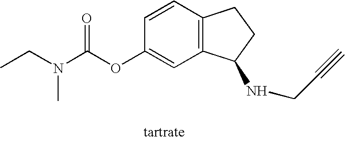 | 300 | >1000 | <0.3 |
| 147 | | 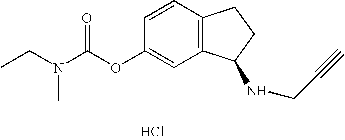 | 550 | >1000 | <0.55 |
| 148 | | 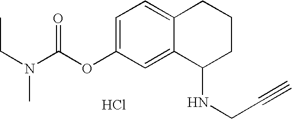 | 500 | 100 | 5 |
| 149 | | 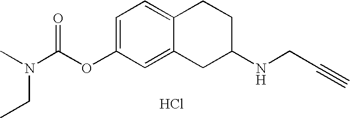 | 2 | 100 | 0.02 |

TABLE 6

| | | | | | % inhi- | |
|---|---|---|---|---|---|---|
| | | dose | | | bition | |
| Cmpd # | structure | mg/kg | μmol/kg | days | A | B |
| 101 | 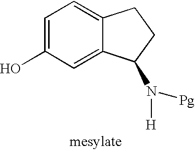 | 5 25 | 18 88 | 10 10 | 35 57 | 47 65 |
| 128 | 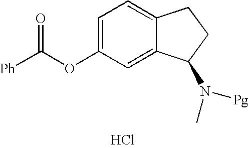 | 1 5 15 | 2.9 15 44 | 7 7 7 | 87 95 98 | 77 91 91 |
| 103 | 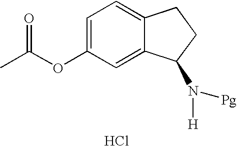 | 5 25 | 18.7 94 | 10 10 | 21 64 | 32 77 |
| 130 | 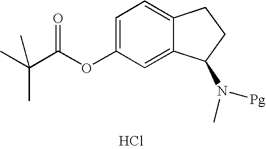 | 3.2 | 10 | acute | 24 | 22 |
| 132 | 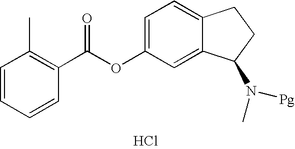 | 3.6 | 10 | acute | 41 | 37 |
| 134 | 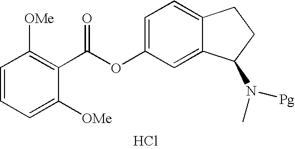 | 4 | 10 | acute | 9 | 0 |
| 146 | 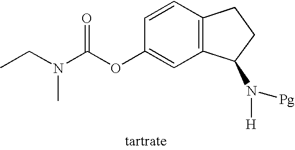 | 17 52 | 50 100 150 | 7 5 7 | 46 56 74 | 52 74 87 |
| 140 | 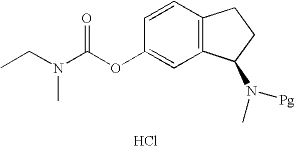 | 0.64 3.2 16.1 | 2 10 50 | 5 5 5 | 6 51 65 | 10 30 73 |
| 150 | 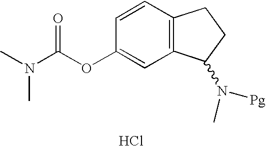 | 0.62 3.1 15.4 | 2 10 50 | 5 5 5 | 7 25 80 | −5 14 73 |

### EXPERIMENTAL DETAILS

### Example 1

General Procedure for Propyn-2-Ylamino (Propargylamino) Indanols (R₃=H)

A mixture of amino indanol(35 mmol), propargyl bromide (35 mmol) and potassium carbonate (35 mmol) in DMA (100 ml) was stirred at room temperature (RT) for 24 hours. The reaction mixture was filtered, diluted with water (200 ml) and extracted with toluene (4×100 ml). The organic extracts were combined, dried and evaporated to dryness under reduced pressure. The residue was then subjected to flash column chromatography (hexane:EtOAc, 1:1). The free base was optionally converted to an acid addition salt.

Alternatively, the propargylation reaction was run in acetonitrile at elevated temperature, e.g., 60° C. for 4 hours. The reaction mixture was then filtered, and the cake washed with acetonitrile. The combined layers were evaporated to dryness, and the residue (brown oil) subjected to flash column chromatography (hexane:EtOAc, 2:1). The product (white solid) was thus obtained in 40-55% yield.

Thus were prepared: (R)-3-prop-2-ynylamino-5-indanol mesylate, (S)-3-prop-2-ynylamino-5-indanol mesylate, 1-prop-2-ynylamino-4-indanol HCl, and 3-prop-2-ynylamino-4-indanol HCl.

### Example 2

General Procedures for N-Methyl-Prop-2-Ynylamino Indanols (R₃=Me), Exemplified by 3-(Methyl-Prop-2-Ynyl Amino)-5-Indanol

Experiment 2A

A mixture of (S)-3-prop-2-ynylamino-5-indanol (5.0 g, 26.7 mmol), paraformaldehyde (3.6 g, 30 mmol) and NaCNBH₃ (1.96 g, 31.2 mmol) in abs MeOH (90 ml) was refluxed under argon for 4 hours. The crude product obtained after evaporation of the solvent was purified by flash chromatography (hexane:EtOAc, 70:30) and was converted to its HCl salt (etheral HCl: 4.2 g(17.6 mmol, 66%)). ¹H NMR (DMSO-d₆):11.7(br d,NH), 9.62(br s, OH), 6.8-7.3(3H), 4.98(m,1H), 3.98(ABq,2H), 3.0(m,1H), 2.90(m,1H), 2.77(s,Me), 2.48(m1H), 2.40(m,1H) ppm.

¹H NMR(D₂O):7.29(d,1H),6.95-7.02(2H),5.09(m, 1H),4.0(AB q, 2H), 3.0(m, 1H),2.90(m, 1H),2.77(s,Me),2.48(m1H),2.40(m, 1H) ppm.

Thus were prepared (R)3-(methyl-prop-2-ynylamino)-5-indanol and 1-(methyl-prop-2-ynylamino)-4-indanol.

### Example 2B

3-(methyl-prop-2-ynylamino)-4-indanol

Experiment 2B1

3-amino-4-indanol (3.70 g, 24.8 mmol) in ethylformate (200 ml) was refluxed for 18 hr. The solvent was then removed under reduced pressure, and the residue was purifed by flash chromatography to give 4.10 g (93%) of N-(7-hydroxy-indan-1-yl)-formamide as a yellow solid.

Experiment 2B2

Lithium aluminium hydride (4.5 g) was added portionwise to stirred and cooled dry THF (100 ml) at 0° C. A solution of N-(7-hydroxy-indan-1-yl)-formamide (4.1 g) in dry THF (70 ml) was added while maintaining the temperature at 5-10° C. The reaction mixture was stirred at ambient temperature for 9 hr, cooled and treated with water (100 ml). The pH was adjusted to 8-9, water (200 ml) was added, and the mixture was extracted with ether (6×300 ml). The etheral extract was evaporated to dryness to give 3.2 g (94%).

Experiment 2B3

3-Methylamino-4-indanol was reacted with propargyl bromide in acetonitrile as described in Example 1.

### Example 2C

7-(methyl-prop-2-ynylamino)-2-tetralinol and 6-(methyl-prop-2-ynylamino)-2,3-tetralindiol were prepared according to Chumpradit et al. and Horn et al.

### Example 3

General Procedure for Esterification of Prop-2-Ynyl Amino Indanols and Teralinols, Exemplified by Pentanoic Acid (R)-3-Prop-2-Ynylamino-Indan-5-Yl Ester HCl(Cmpd # 107)

To a solution of (R)3-prop-2-ynylamino-5-indanol (2.5 g, 13.4 mmol) in CHCl₃(30 ml) and TFA (5 ml), was added valeryl chloride (2.03 g, 2.0 ml, 16.7 mmol) . The solution was heated at 600 for 8 hours and cooled to RT. Water (250 ml) was added, and the pH adjusted to 7 by means of concentrated aqueous ammonia. Extracted with methylene chloride (4×100 ml), dried and evaporated to dryness under reduced pressure. The residue (brown oil, 3.65 g) was purified by flash chromatography (SiO₂, CH₂Cl₂:MeOH 99:1). The free base thus obtained (3.25 g) was dissolved in dry ether (80 ml), and 20% etheral HCl was added. The resulting suspension was stirred for 2 hours at RT, the solid product was collected by filtration and washed with ether (20 ml) and dried at 60° to give 3.45 g (11.2 mmol, 85%) of the ester HCl.

### Example 4

Alternative Procedure, Exemplified by Benzoic Acid (R)-3-Prop-2-Ynylamino-Indan-5-Yl Ester (Cmpd # 111)

(R) 3-prop-2-ynylamino-5-indanol (3.0 g, 16 mmol) was dissolved in dry THF (75 ml), and triethylamine (3.15 ml, 22.6 mmol) followed by Boc₂O(4.5 g, 20.6 mmol) was added. The solution was stirred at RT for 24 hours and evaporated to dryness. The residue was taken up in water (200 ml) and extracted with CH₂Cl₂(4×100 ml). The organic layers were combined, dried and evaporated to dryness. The crude product was purified by flash column chromatography (hexane:EtOAc 3:1) to give 3.75 g (81.5%) of a white solid.

¹H NMR (DMSO-d6)(a 1:1 mixture of 2 rotamers): 9.17(s,OH), 7.0 (d,1H), 6.62 (dd, 1H), 6.5(br s, 1H), 5.51 & 5.22 (brs, 1H), 4.05, 3.72, 3.60, 3.38(m,2H), 3.06(br s, 1H), 2.83 (m,1H), 2.64(m,1H),2.30(br s, 1H, 2.10(br s, 1H), 1.4 & 1.27 (2s,9H) ppm.

(R) N-Boc 3-prop-2-ynylamino-5-indanol(2.65 g, 9.23 mmol) was dissolved in dry methylene chloride (20 ml), and triethylamine (2.65 ml, 18.5, mmol), DMAP (0.11 g, 0.9 mmol) and benzoyl chloride (1.7 ml, 18.5 mmol) was added. The solution was stirred at RT for 3 hours, water (100 ml) was added and acidified to pH 4 (aq HCl). The organic layer was separated and washed with 10% HCl. The aqueous layer was washed with methylene chloride (100 ml), and the combined organic phases were dried and evaporated to dryness in vacuo. The crude product (5.2 g brown oil) was purified by flash column chromatography (hexane:EtOAc 3:1) to give 4.1 g (90%) of a white solid.

(R) N-Boc-3-prop-2-ynylamino-5-benzoyloxy indan (2.55 g, 6.5 mmol) was dissolved in dioxan (25 ml), and HCl/dioxan (25 ml) was added. The mixture was stirred at RT for 4 hours and the solvent was evaporated to dryness in vacuo. Ether (50 ml) was added, the suspension was then stirred at RT for 2 hours. The solid was collected by filtration, washed with ether and dried (1.5 g). The crude product was crystallized from iPrOH (90 ml) to give 1.3 g (3.96 mmol, 61%), mp 210-2° C.

### Example 5

Preparation of Benzoic Acid (S)-3-(Methyl-Prop-2-Ynyl-Amino)-Indan-5-Yl Ester HCl Cmpd # 127

(S)3-(methyl-prop-2-ynylamino)-5-indanol (1.5 g, 7.46 mmol) was dissolved in dry methylene chloride (15 ml), and triethylamine (2.15 ml, 15.5 mmol), DMAP (0.08 g, 0.66 mmol) and benzoyl chloride (2.1 ml, 18.1 mmol) was added. The solution was stirred at RT for 2 hours, water (100 ml) was added and acidified to pH 4 (aq HCl). The organic layer was separated, washed with 10% HCl. The aqueous layer was washed with methylene chloride (4×100 ml), and the combined organic phases were dried evaporated to dryness in vacuo. The crude product (3.78 g brown oil) was purified by flash column chromatography (hexane:EtOAc 4:1) to give 1.6 g (5.3 mmol, 71%) of a yellow oil. The free base was converted to the HCl salt (etheral HCl, 2 hours, RT), 1.39 g (4.07 mmol, 77%, 55% from the hydroxy compound).

By the same procedure was prepared 7-O-benzoyl-2-(methyl-prop-2-ynylamino)-tetralin Hcl, ¹HNMR (D₂0): 7.20, 6.98, 6.95 (3H, ArOCO), 8.05, 7.71, 7.53 (5H, PhCOO), 4.15 (m, 2H, CH₂CCH), 3.80 (m, 1H, C₇—H), 3.15 (t, 1H, CH₂CCH), 3.14, 3.01 (m, 2H, C₈—H), 2.8-3.0 (m, 2H, C₅—H), 2.31, 1.87 (m, 2H, C₆—H), 3.0 (S, 3H, Me) ppm.

The same procedure was also used to prepare 6,7-di-O-benzoyl-2-(methyl-prop-2-ynylamino)-tetralin HCl, ¹HNMR (DM80-d₆): 7.91 (dd, 4H), 7.65 (t,2H), 7.46 (t,4H), 7.28 (s, 2H), 4.24 (br s, 2H), 3.87 (br s, 1H), 3.74 (m, 1H), 3.35-2.90 (m, 4H), 2.87 (s, 3H), 2.39 (m, 1H), 1.90 (m, 1H) ppm.

### Example 6

Preparation of 5,6-Di-O-Benzoyl-1-(Methyl-Prop-2-Ynyl-Amino)-Indan HCl Cmpd # 137):

Experiment 6A: (5,6-Bis-benzyloxy-1-indan-1-yl)-methylamine HCl (FIG. 2, Compound 4)

A mixture of 5,6-dibenzyloxy-1-indanone (10.0 g, 29 mmol), 8M ethanolic methylamine (30 ml, 240 mmol), methylamine HCl (7.15 g, 106 mmol), and NaCNBH₃ (2.95 g, 47 mmol) in dry THF (750 ml) and methanol (250 ml) was refluxed under nitrogen for 4 hours. The reaction mixture was cooled to 5° C., acidified with concentrated HCl to pH 1.5, and evaporated to dryness. The solid residue was treated with a mixture of methylene chloride (600 ml) and water (400 ml). The aqueous layer was separated, extracted with methylene chloride (4×100 ml), and the combined organic layers were evaporated to dryness. The crude product thus obtained was slurried in EtOAc (80 ml) for 30 min at RT, filtered and purified by flash column chromatography (CH₂Cl₂ :MeOH, 80:20); to give 6.3 g (54.8%), mp: 180-182° C.

Experiment 6B: 1-Methylamino-1-indan-5,6-diol HCl (FIG. 2, Compound 5)

A solution of (5,6-bis-benzyloxy-1-indan-1-yl)-methylamine HCl (3.15 g, 7.96 mmol) in MeOH (250 ml) was hydrogenated (44 psi) over 10% Pd/C (1.05 g) at RT for 3 hours. The mixture was filtered (Filteraid), and the filtrate evaporated to dryness. The residue was treated with charcoal in boiling MeOH, filtered and evaporated to dryness, to give 1.6 g of a light grey solid, mp: 153-5° C.

¹H NMR (DM80-d₆): 9.3-8.8 (3H, br m, OH, NH₂), 7.02 (s, 1H, Ar), 6.08 (s, 1H, Ar), 4.7 (dd, 1H, C₃—H), 2.92 (m, 1H, C₁—H), 2.66 (m, 1H, C₁—H), 2.44 (s, 3H, Me), 2.33 (m, 1H, C₂—H), 2.11 (m, 1H, C₂—H′) ppm.

Experiment 6C: N-Boc-1-methylamino-1-indan-5,6-diol (FIG. 2, Compound 6)

To a solution of 1-methylamino-1-indan-5,6-diol HCl (0.5 g, 2.32 mmol) in water (30 ml) was added dioxane (30 ml), NaHCO₃ (0.6 g) and Boc₂O (0.6 g). The reaction mixture was stirred at RT for 4 hours under nitrogen, evaporated to dryness, and the solid residue taken up in a mixture of water (100 ml) and methylene chloride (100 ml). The aqueous layer was separated and extracted with methylene chloride (5×50 ml). The latter was filtered, washed with water, dried and evaporated to dryness to give a viscous oil which was purified by flash column chromatography (CH₂Cl₂:MeOH, 95:5) to give 0.35 g (54%) of a viscous oil which soon solidified.

Experiment 6D: N-Boc-(5,6-di-O-benzoyl-1-indan-1-yl)-methylamine (FIG. 2, Compound 7)

To a solution of N-Boc-1-methylamino-1-indan-5,6-diol (0.34 g, 1.22 mmol) in methylene chloride (15 ml) was added triethylamine (0.49 g, 4.88 mmol), DMAP (0.03 g, 0.244 mmol) and benzoyl chloride (0.69 g, 4.88 mmol), and the solution was stirred at RT for 4.5 hours. Water (100 ml) was added, acidified to pH 4 with dilute HCl. The organic layer was separated and washed with 10% HCl. The aqueous layer was extracted with methylene chloride (2×75 ml), and the latter was washed with 10% HCl. The combined organic layers were dried, evaporated to dryness, and the residue purified by flash column chromatography (hexane:EtOAc, 50:50) to give 0.50 g (40%) of a yellow oil.

Experiment 6E: (5,6-di-O-Benzoyl-1-indan-1-yl)-methylamine HCl (FIG. 2, Compound 8)

To a solution of N-Boc-(5,6-di-O-benzoyl-1-indan-1-yl)-methylamine (0.29 g, 0.59 mmol) in dioxane (5 ml) was added 20% HCl in dioxane (5 ml), and the mixture stirred at RT for 4 hours under nitrogen. The solvent was removed and ether (40 ml) was added to the residue, and the suspension stirred at RT for 1 hour. The solvent was removed to give 0.11 g (89%) of a white solid, mp: 192-3° C.

¹H NMR (CDCl₃): 8.1-7.2 (12H, Ar), 4.79 (br s, 1H, C₃—H), 3.40 (m, 1H, C₁—H), 3.01 (m,1H,C₁—H), 2.60 (s, 3H, Me), 2.50 (m, 1H, C₂—H), 1.83 (m, 1H, C₂—H′) ppm.

Experiment 6F: 5,6-di-O-Benzoyl-1-(methyl-prop-2-ynyl-amino)-indan HCl (FIG. 2, Compound 9 (Cmpd # 137))

To a solution of (5,6-di-O-benzoyl-1-indan-1-yl)-methylamine HCl (˜0.2 g, 0.48 mmol) in acetonitrile (100 ml) was added K₂CO₃ (130 mg, 0.96 mmol), followed after 15 min by a solution of propargyl bromide (56 mg, 0.48 mmol) in acetonitrile (10 ml). The reaction mixture was stirred under nitrogen at RT for 20 hours, filtered and evaporated to dryness. The crude product was purified by flash column chromatography (hexane:EtOAc, 50:50) to give 0.15 g (0.35 mmol, 75%) of a viscous light tan oil.

The free base was dissolved in MeOH (30 ml), and saturated etheral HCl (4 ml) was added. The solution was stirred at RT for 30 min and evaporated to dryness. The oily residue was triturated three times in ether, to give 120 mg (0.26 mmol, 74%) of a light tan solid.

NMR (CDCl₃): 8.1-7.2 (m, 12H, Ar), 5.1 (br d, 1H, C1-H), 3.91 (br s, 2H, CH₂CCH), 3.6-2.5 (m, 8H, indan CH₂'s, Me, CH₂CCH).

### Example 7

Inhibition of MAO Activity In Vitro Experimental Protocol

The MAO enzyme source was a homogenate of rat brain in 0.3 M sucrose 1:20 w/v. The homogenate was pre-incubated with serial dilutions of the test compounds (Table 5) for 60 minutes at 37° C. ¹⁴C-labeled substrates (2-phenylethylamine, hereinafter PEA; 5-hydroxytryptamine, hereinafter 5-HT) were then added, and the incubation continued for a further 20 minutes (PEA), or 30-45 minutes (5-HT). In the case of PEA, the enzyme concentration was chosen so that not more than 10% of the substrate was metabolized during the course of the reaction. The reaction was then stopped by addition of citric acid. Radioactivity indicates the production of 5-HT and PEA metabolites formed as a result of MAO activity. Activity of MAO in the sample was expressed as a percentage of control activity in the absence of test compounds after subtraction of appropriate blank values. The activity determined using PEA as substrate is referred to as MAO-B, and that determined using 5-HT as MAO-A.

### Example 8

Inhibition of MAO Activity In Vivo: Chronic Treatment Experimental Protocol

Rats were treated with the test compounds (Table 5) at several dose levels by oral administration, one dose daily for 7-21 days, and decapitated 2 hours after the last dose. The activities of MAO-A and MAO-B were determined in the brain, liver and intestine as described in the previous example. Inhibition of MAO activity was calculated by dividing MAO activity in the treated rats by MAO activity in the control rats (saline treated, MAO activity in these rats was taken as 100%).

A mixture of toluene: ethyl acetate (1:1) was added to the reaction and mixed for 10 minutes, followed by 5 minutes of centrifugation at 1760 g. The upper phase was taken for radioactive determination by liquid scintillation spectrometry.

### REFERENCES

U.S. Pat. No. 3,513,244, Gittos et al., issued May 19, 1970.

U.S. Pat. No. 4,844,033, Abinett et al., issued Jul. 4, 1989.

U.S. Pat. No. 5,387,612, Youdim et al., issued Feb. 7, 1995.

U.S. Pat. No. 5,453,446, Youdim et al., issued Sep. 26, 1995.

U.S. Pat. No. 5,457,133, Youdim et al., issued Oct.10, 1995.

U.S. Pat. No. 5,519,061, Youdim et al., issued May 21, 1996.

U.S. Pat. No. 5,532,415, Youdim et al., issued Jul. 2, 1996.

U.S. Pat. No. 5,576,353, Youdim et al., issued Nov. 19, 1996.

U.S. Pat. No. 5,649,913, Cohen, issued Jul. 22, 1997.

U.S. Pat. No. 5,668,181, Youdim et al., issued Sep. 16, 1997.

U.S. Pat. No. 5,744,500, Youdim et al., issued Apr. 28, 1998.

U.S. Pat. No. 5,786,390, Youdim et al., issued Jul. 28, 1998.

U.S. Pat. No. 5,844,003, Tatton, issued Dec. 1, 1998.

U.S. Pat. No. 5,877,221, Cohen et al., issued Mar. 2, 1999.

U.S. Pat. No. 5,877,218, Herzig et al., issued Mar. 2, 1999.

U.S. Pat. No. 5,880,159, Herzig et al., issued Mar. 9, 1999.

U.S. Pat. No. 5,891,923, Youdim et al., issued Apr. 6, 1999.

U.S. Pat. No. 5,914,349, Cohen et al., issued Jun. 22, 1999.

U.S. Pat. No. 5,994,408, Cohen et al., issued Nov. 30, 1999.

U.S. Pat. No. 6,303,650, Chorev et al., issued Oct. 16, 2001.

U.S. Pat. No. 6,316,504, Youdim et al., issued Nov. 13, 2001.

PCT International Application No. PCT/IL96/00115, Berger et al., published Apr. 10, 1997.

PCT International Application No. PCT/US95/00245, Cohen et al., published Jul. 13, 1995.

PCT International Application No. PCT/US97/24155, Chorev et al., published Jun. 25, 1998.

European Patent No. 0 436 492, Youdim et al., issued Jun. 8, 1994.

Bentue-Ferrer et al., CNS Drugs 6, 217, 1996.

Chrisp et al., Drugs & Aging, 1, 228, 1991.

Chumpradit, S. et al., J. Med. Chem., 36, 4308, 1993.

Dostert, J. Neurol. Transm. Suppl 41, 269, 1994.

Fitton et al., Drugs, 43, 561, 1992.

Florvall et al., Eur. J. Med. Chem. 34, 137, 1999.

Kleywegt, Monoamine oxidase, 1999 (http://alpha2.bmc.uu.se/gerard/mao.html).

Krageten et al., J. Biol. Chem., 273, 5821, 1998.

Hazelhoff et al., Naunyn-Schmeideberg's Arch. Pharmacol., 1985, 330, 50.

Hazelhoff, B. et al, Eur. J. Pharmacol., 109, 229, 1985.

Horn, A. S. et al., J. Med. Chem., 25, 993, 1982.

Lidor et al., 1997, Organic Preparations and Procedures International (OPPI) 29,701.

Loscher et al., J. Pharmacol and Expt. Therap. 288, 984, 1999.

Palfreyman et al., J. Neurochemistry, 45, 1850, 1985.

Szelenyi, I., Inhibitors of Monoamine Oxidase B: Pharmacology and Clinical Use in Neurodegenerative Disorders, 1992,

Bierkhauser Berlag, Switzerland.

White et al., Drug Development Research, 25, 191, 1992.

Youdim et al., Handbook of Experimental Pharmacology, Trendelenburg and Weiner, eds. Springer-Verlag, 1988, 90, ch.3.

The interaction of L-deprenyl and scopolamine on spatial learning/memory in rats, J. Neural Trans. Parkinson's Dis. Dementia Sect., 1993, 6(3): 189-197.

Potential applications for monoamine oxidase B inhibitors, Dementia, 1990, 1(6): 323-348.

Treatment of Tourette's: An Overview, (http://www.haverford.edu/psych/biopsych217b/tourettes/TSwebtreat.caf.html).

What is claimed is:

1. A compound having the structure: 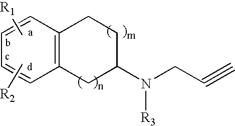
wherein R₁ is OC(O)R₉ and R₂ is H,
wherein R₉ is branched or unbranched C₁ to C₆ alkyl, aryl, or aralkyl, or
R₁ is OC(O)R₄ and R₂ is OC(O)R₄,
wherein R₄ is branched or unbranched C₁ to C₆ alkyl, aryl, aralkyl or NR₅R₆,
wherein R₅ and R₆ are each independently H, C₁ to C₈ alkyl, C₆ to C₁₂ aryl, C₆ to C₁₂ aralkyl or C₆ to C₁₂ cycloalkyl, each optionally substituted;
wherein R₃ is H or C₁ to C₆ alkyl;
wherein n is 0 or 1; and
wherein m is 1 or 2,
or a pharmaceutically acceptable salt thereof.

2. The compound of claim 1, wherein the pharmaceutically acceptable salt is the acetate salt, mesylate salt, esylate, tartarate salt, hydrogen tartarate salt, benzoate salt, phenylbutyrate salt, phosphate salt, citrate salt, ascorbate salt, mandelate salt, adipate salt, octanoate salt, the myristate salt, the succinate salt, or fumarate salt.

3. The compound of claim 1 having the structure:

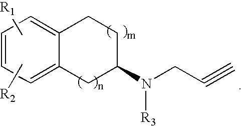

4. The compound of claim 1 having the structure:

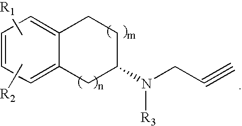

5. The compound of claim 1 having the structure:

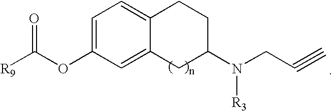

6. The compound of claim 5, wherein n is 1.

7. The compound of claim 6 having the structure:

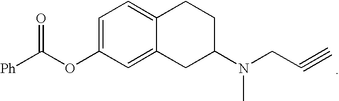

8. The compound of claim 5, wherein n is 0.

9. The compound of claim 8 having the structure:

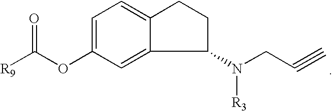

10. The compound of claim 8 having the structure:

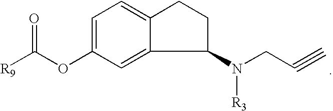

11. The compound of claim 8, wherein R₉ is Me and R₃ is H.

12. The compound of claim 8, wherein R₉ is tBu and R₃ is H.

13. The compound of claim 8, wherein R₉ is nBu and R₃ is H.

14. The compound of claim 8, wherein R₉ is CH₂Ph and R₃ is H.

15. The compound of claim 8, wherein R₉ is Ph and R₃ is H.

16. The compound of claim 8, wherein R₉ is Me and R₃ is Me.

17. The compound of claim 8, wherein R₉ is nBu and R₃ is Me.

18. The compound of claim 8, wherein R₉ is Ph and R₃ is Me.

19. The compound of claim 8, wherein R₉ is tBu and R₃ is Me.

20. The compound of claim 8, wherein R₉ is Ph(Me) and R₃ is Me.

21. The compound of claim 8, wherein R₉ is Ph(OMe)2 and R₃ is Me.

22. The compound of claim 8, wherein R₉ is Ph(OMe)₂ and R₃ is H.

23. The compound of claim 1 having the structure:

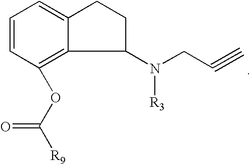

24. The compound of claim 23, wherein R₃ is Me and R₉ is Me.

25. The compound of claim 23, wherein R₃ is Me and R₉ is Ph.

26. The compound of claim 23, wherein R₃ is Me and R₉ is Ph(OMe)₂.

27. The compound of claim 1 having the structure:

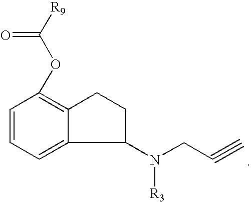

28. The compound of claim 27, wherein R₃ is Me and R₉ is Me.

29. The compound of claim 27, wherein R₃ is H and R₉ is Ph.

30. The compound of claim 27, wherein R₃ is H and R₉ is Ph(OMe)₂.

31. The compound of claim 1 having the structure:

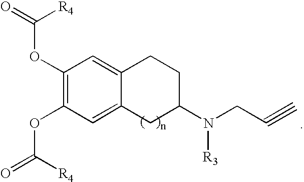

32. The compound of claim 31, wherein n is 0.

33. The compound of claim 32, wherein R₄ is Ph and R₃ is Me.

34. The compound of claim 31, wherein n is 1.

35. The compound of claim 34, wherein R₃ is Me.

36. The compound of claim 31 having the structure:

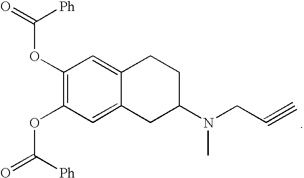

37. A compound having the structure: 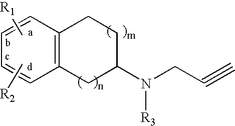
wherein R₁ is OH;
wherein R₂ is H or OC(O)R₄ when R₁ is attached to the “a” carbon or the “d” carbon, or
R₂ is OC(O)R₄ when R₁ is attached to the “b” carbon or the “c” carbon;.
wherein R₄ is C₁ to C₆ branched or unbranched alkyl, aryl, aralkyl or NR₅R₆,
wherein R₅ and R₆are each independently H, C₁ to C₈ alkyl, C₆ to C₁₂ aryl, C₆ to C₁₂ aralkyl or C₆ to C₁₂ cycloalkyl, each optionally substituted;
wherein n is 0 or 1, and m is 1 or 2; and
wherein R₃ is H or Me when n is 1 and m is 1, or R₃ is H or C₁ to C₆ alkyl when n is 0 or m is 2,
or a pharmaceutically acceptable salt thereof.

38. The compound of claim 37, wherein the pharmaceutically acceptable salt is the acetate salt, mesylate salt, esylate, tartarate salt, hydrogen tartarate salt, benzoate salt, phenylbutyrate salt, phosphate salt, citrate salt, ascorbate salt, mandelate salt, adipate salt, octanoate salt, the myristate salt, the succinate salt, or fumarate salt.

39. The compound of claim 37 having the structure:

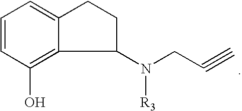

40. The compound of claim 39, wherein R₃ is H.

41. The compound of claim 39, wherein R₃ is Me.

42. The compound of claim 37 having the structure:

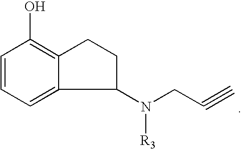

43. The compound of claim 42, wherein R₃ is H.

44. The compound of claim 42, wherein R₃ is Me.

45. A compound having the structure: 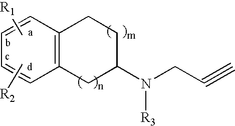
wherein the compound is an optically pure enantiomer;
wherein R₁ is OH;
wherein R₂ is H;
wherein R₃ is H or C₁ to C₆ alkyl;
wherein n is 0 or 1; and
wherein m is 1 or 2,
or a pharmaceutically acceptable salt thereof.

46. The compound of claim 45, wherein the pharmaceutically acceptable salt is the acetate salt, mesylate salt, esylate, tartarate salt, hydrogen tartarate salt, benzoate salt, phenylbutyrate salt, phosphate salt, citrate salt, ascorbate salt, mandelate salt, adipate salt, octanoate salt, the myristate salt, the succinate salt, or fumarate salt.

47. The compound of claim 45 having the structure:

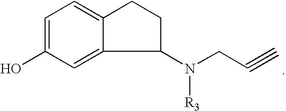

48. The compound of claim 47 having the structure:

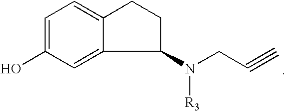

49. The compound of claim 48, wherein R₃ is H.

50. The compound of claim 48, wherein R₃ is Me.

51. The compound of claim 47 having the structure:

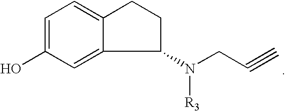

52. The compound of claim 51, wherein R₃ is H.

53. The compound of claim 51, wherein R₃ is Me.

54. A compound having the structure: 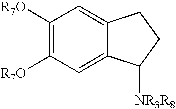
wherein R₇ is H, C₁ to C₆ alkyl, aryl, aralkyl or C(O)R₄,
wherein R₄ is branched or unbranched C₁ to C₆ alkyl, aryl, aralkyl or NR₅R₆,
wherein R₅ and R₆are each independently H, C₁ to C₈ alkyl, C₆ to C₁₂ aryl, C₆ to C₁₂ aralkyl or C₆ to C₁₂ cycloalkyl, each optionally substituted;
wherein R₃ is H or C₁ to C₆ alkyl;
wherein R₈ is H or t-butoxycarbonyl (Boc).

55. The compound of claim 54 having the structure:

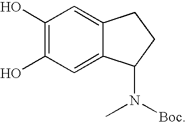

56. The compound of claim 54 having the structure:

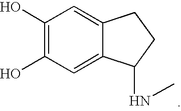

57. The compound of claim 54 having the structure:

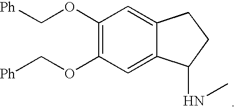

58. The compound of claim 54 having the structure:

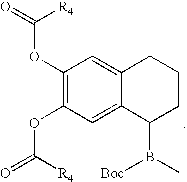

59. The claim 58, wherein R₄ is Ph.

60. The compound of claim 54 having the structure:

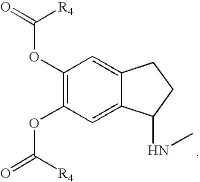

61. The claim 60, wherein R₄ is Ph.

62. A pharmaceutical composition comprising the compound of claim 1 or a pharmaceutically acceptable salt thereof, and a pharmaceutically acceptable carrier.

63. A pharmaceutical composition comprising the compound of claim 37 or a pharmaceutically acceptable salt thereof, and a pharmaceutically acceptable carrier.

64. A pharmaceutical composition comprising the compound of claim 45 or a pharmaceutically acceptable salt thereof, and a pharmaceutically acceptable carrier.

65. A method of treating a subject afflicted with a neurological disease comprising administering to the subject a compound having the structure: 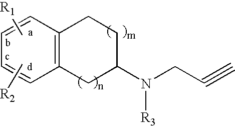
wherein R₁ is OH or OC(O)R₄;
wherein R₂ is H, OH or OC(O)R₄,
wherein R₄ is branched or unbranched C₁ to C₆ alkyl, aryl, aralkyl or NR₅R₆,
wherein R₅ and R₆are each independently H, C₁ to C₈ alkyl, C₆ to C₁₂ aryl, C₆ to C₁₂ aralkyl or C₆ to C₁₂ cycloalkyl, each optionally substituted;
wherein R₃ is H or C₁ to C₆ alkyl;
wherein n is 0 or 1; and
wherein m is 1 or 2,
or a pharmaceutically acceptable salt thereof, or a prodrug which becomes the compound in the subject, so as to thereby treat the neurological disease in the subject.

66. A method of treating a subject afflicted with a neurological disease comprising administering to the subject a compound having the structure: 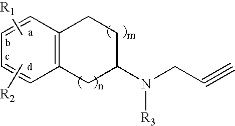
wherein R₁ is OH or OC(O)R₉, and wherein R₉ is branched or unbranched C₁ to C₆ alkyl, aryl, or aralkyl;
R₂ is H or OC(O)R₄, or both R₁ and R₂ are OC(O)R₄,
wherein R₄ is branched or unbranched C₁ to C₆ alkyl, aryl, aralkyl or NR₅R₆,
wherein R₅ and R₆are each independently H, C₁ to C₈ alkyl, C₆ to C₁₂ aryl, C₆ to C₁₂ aralkyl or C₆ to C₁₂ cycloalkyl, each optionally substituted;
wherein R₃ is H or C₁ to C₆ alkyl;
wherein n is 0 or 1; and
wherein m is 1 or 2,
or a pharmaceutically acceptable salt thereof, or a prodrug which becomes the compound in the subject, so as to thereby treat the neurological disease in the subject.

67. The method of claim 66, wherein the compound has the structure: 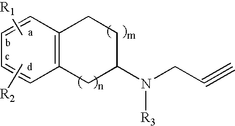
wherein R₁ is OC(O)RG and R₂ is H,
wherein R₉ is branched or unbranched C₁ to C₆ alkyl, aryl, or aralkyl, or
R₁ is OC (O) R₄ and R₂ is OC(O)R₄,
wherein R₄ is branched or unbranched C₁ to C₆ alkyl, aryl, aralkyl or NR₅R₆,
wherein R₅ and R₆ are each independently H, C₁ to C₈ alkyl, C₆ to C₁₂ aryl, C₆ to C₁₂ aralkyl or C₆ to C₁₂ cycloalkyl, each optionally substituted;
wherein R₃ is H or C₁ to C₆ alkyl;
wherein n is 0 or 1; and
wherein m is 1 or 2.

68. The method of claim 66, wherein the compound has the structure: 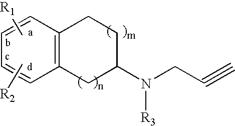
wherein R₁ is OH;
wherein R₂ is H or OC(O)R₄ when R₁ is attached to the “a” carbon or the “d” carbon, or
R₂ is OC(O) R₄ when R₁ is attached to the “b” carbon or the “c” carbon;
wherein R₄ is C₁ to C₆ branched or unbranched alkyl, aryl, aralkyl or NR₅R₆,
wherein R₅ and R₆ are each independently H, C₁ to C₈ alkyl, C₆ to C₁₂ aryl, C₆ to C₁₂ aralkyl or C₆ to C₁₂ cycloalkyl, each optionally substituted;
wherein R₃ is H or C₁ to C₆ alkyl;
wherein n is 0 or 1; and
wherein m is 1 or 2.

69. The method of claim 66, wherein the compound has the structure: 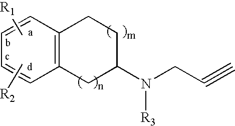
wherein the compound is an optically pure enantiomer;
wherein R₁ is OH;
wherein R₂ is H;
wherein R₃ is H or C₁ to C₆ alkyl;
wherein n is 0 or 1; and
wherein m is 1 or 2.

70. The method of claim 66, wherein the subject is human.

71. The method of claim 66, wherein the administration comprises oral, parenteral, intravenous, transdermal, or rectal administration.

72. The method of claim 66, wherein the effective amount is from about 0.01 mg per day to about 50.0 mg per day.

73. The method of claim 66, wherein the effective amount is from about 0.1 mg per day to about 100.0 mg per day.

74. The method of claim 73, wherein the effective amount is from about 0.1 mg per day to about 10.0 mg per day.

75. The method of claim 66, wherein the neurological disease is Parkinson's disease, Alzheimer's disease, depression, epilepsy, narcolepsy, amyotrophic lateral sclerosis (ALS), memory disorders, panic, post-traumatic stress disorder (PTSD), sexual dysfunction, attention deficit and hyperactivity syndrome (ADHD), attention deficit disorder, or Tourette's syndrome.

76. The method of claim 75, wherein the neurological disease is depression.

77. The method of claim 75, wherein the compound has the structure:

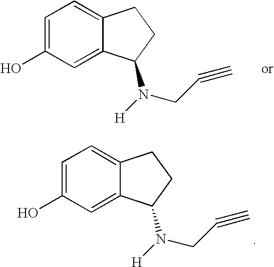

78. A process for preparing a compound having the structure: 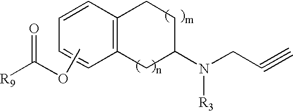
wherein n is 0 or 1, and m is 1 or 2;
wherein R₃ is H or C₁ to C₆ alkyl; and
wherein R₉ is branched or unbranched C₁ to C₆ alkyl, aryl, or aralkyl;
comprising the step of reacting 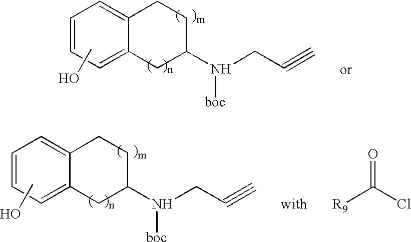
in the presence of an acid or 4-dimethylaminopyridine (DMAP) to form the compound.

79. The process of claim 78 for preparing a compound having the structure: 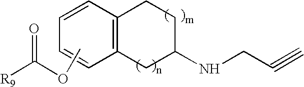
wherein R₉ is branched or unbranched C₁ to C₆ alkyl, aryl, or aralkyl;
which process comprises:
(a) reacting a compound having the structure: 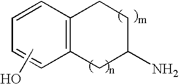
with a compound having the structure: 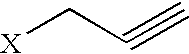
wherein X is a leaving group,
to produce a compound having the structure: 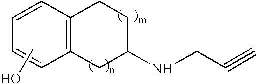
(b) reacting the compound formed in step (a) with a compound having the structure: 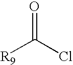
in the presence of trifluoroacetic acid (TFA) and an aprotic solvent to produce a compound having the structure:

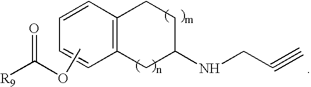

80. The process of claim 79, wherein the leaving group in step (a) is selected from the group consisting of a halogen and benzene sulfonate and the aprotic solvent in step (b) is CHCl₃.

81. The process of claim 78 for preparing a compound having the structure: 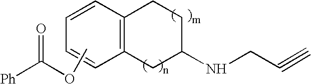
which comprises:
(a) reacting a compound having the structure: 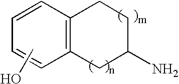
with a compound having the structure: 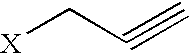
wherein X is a leaving group,
to produce a compound having the structure: 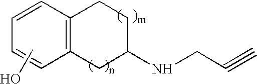
(b) N-protecting the compound formed in step (a) with tert-butoxycarbonyl (Boc) to produce a compound having the structure: 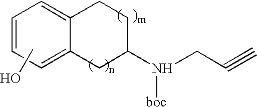
(c) reacting the compound formed in step (b) with a compound having the structure: 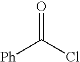
in the presence of 4-dimethylaminopyridine (DMAP) to produce a compound having the structure: 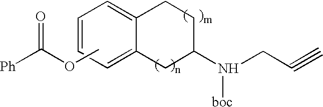
(d) deprotecting the compound formed in step (c) with HCl to produce a compound having the structure:

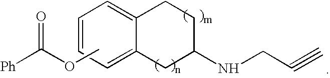

82. The process of claim 81, wherein the leaving group in step (a) is selected from the group consisting of a halogen and benzene sulfonate and the aprotic solvent in step (b) is CHCl₃.

83. The process of claim 78 for preparing a compound having the structure: 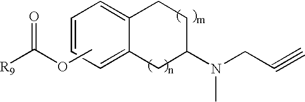
wherein R₉ is branched or unbranched C₁ to C₆ alkyl, aryl, or aralkyl;
which process comprises:
(a) reacting a compound having the structure: 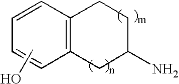
with a compound having the structure: 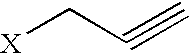
wherein X is a leaving group, to produce a compound having the structure: 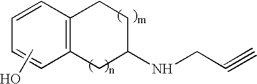
(b) reacting the compound formed in step (a) with NaCNBH₃ and paraformaldehyde to produce a compound having the structure: 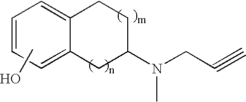
(c) reacting the compound formed in step (b) with a compound having the structure: 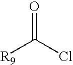
in the presence of trifluoroacetic acid (TFA) and an aprotic solvent to form a compound having the structure:

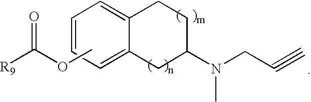

84. The process of claim 83, wherein the leaving group in step (a) is selected from the group consisting of a halogen and benzene sulfonate and the aprotic solvent in step (c) is CHCl₃.

85. The process of claim 78 for preparing a compound having the structure: 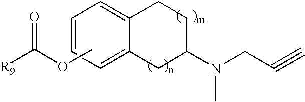
wherein R₉ is branched or unbranched C₁ to C₆ alkyl, aryl, aralkyl or NR₅R₆;
which process comprises:
(a) reacting a compound having the structure: 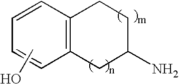
with ethyl formate to produce a compound having the structure: 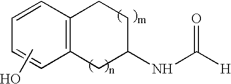
(b) reacting the compound formed in step (a) with lithium aluminum hydride to produce a compound having the structure: 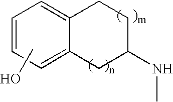
(c) reacting the compound formed in step (b) with a compound having the structure: 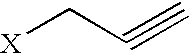
wherein X is a leaving group, to form a compound having the structure: 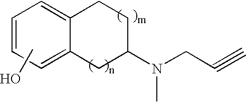
(d) reacting the compound formed in step (c) with a compound having the structure: 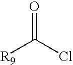
in the presence of trifluoroacetic acid (TFA) and an aprotic solvent to form a compound having the structure:

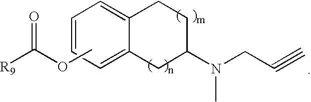

86. The process of claim 85, wherein the aprotic solvent in step (c) is CHCl₃.

87. The process of claim 78 for preparing a compound having the structure: 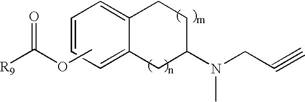
wherein R₉ is branched or unbranched C₁ to C₆ alkyl, aryl, or aralkyl;
which process comprises:
(a) reacting a compound having the structure: 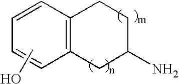
with NaCNBH₃/paraformaldehyde to produce a compound having the structure: 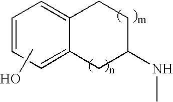
(b) reacting the compound formed in step (a) with a compound having the structure: 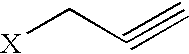
wherein X is a leaving group,
to form a compound having the structure: 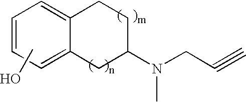
(c) reacting the compound formed in step (b) with a compound having the structure: 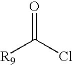
in the presence of trifluoroacetic acid (TFA) and an aprotic solvent to form a compound having the structure:

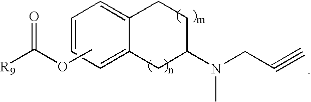

88. The process of claim 87, wherein the aprotic solvent in step (d) is CHCl₃.

89. The process of claim 78 for preparing a compound having the structure: 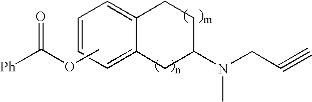
which comprises:
(a) reacting a compound having the structure: 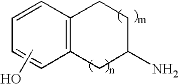
with a compound having the structure: 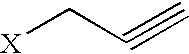
wherein X is a leaving group,
to produce a compound having the structure: 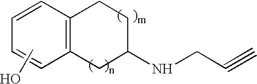
(b) reacting the compound formed in step (a) with NaCNBH₃ and paraformaldehyde to produce a compound having the structure: 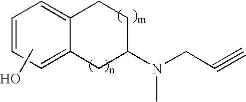
(c) reacting the compound formed in step (b) with a compound having the structure: 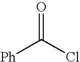
in the presence of 4-dimethylaminopyridine (DMAP) and an aprotic solvent to form a compound having the structure:

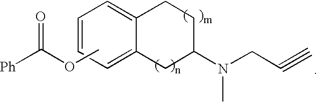

90. The method of claim 89, wherein the leaving group in step (a) is selected from the group consisting of a halogen and benzene sulfonate and the aprotic solvent in step (c) is CHCl₃.

91. The process of claim 78 for preparing a compound having the structure: 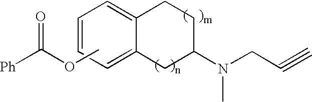
which comprises:
(a) reacting a compound having the structure: 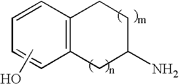
with ethyl formate to produce a compound having the structure: 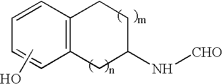
(b) reacting the compound formed in step (a) with lithium aluminum hydride to produce a compound having the structure: 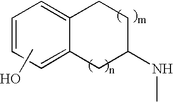
(c) reacting the compound formed in step (b) with a compound having the structure: 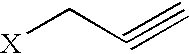
wherein X is a leaving group,
to form a compound having the structure: 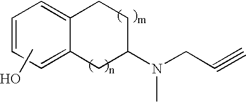
(d) reacting the compound formed in step (c) with a compound having the structure: 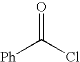
in the presence of 4-dimethylaminopyridine (DMAP) and an aprotic solvent to form a compound having the structure:

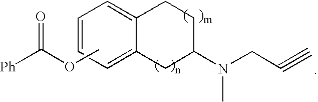

92. The process of claim 91, wherein the aprotic solvent in step (c) is CHCl₃.

93. The process of claim 78 for preparing a compound having the structure: 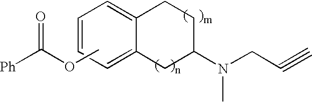
which comprises:
(a) reacting a compound having the structure: 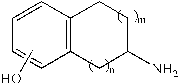
with NaCNBH₃/paraformaldehyde to produce a compound having the structure: 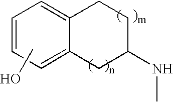
(b) reacting the compound formed in step (a) with a compound having the structure: 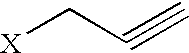
wherein X is a leaving group,
to form a compound having the structure: 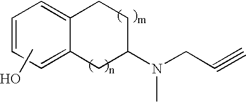
(c) reacting the compound formed in step (b) with a compound having the structure: 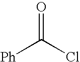
in the presence of 4-dimethylaminopyridine (DMAP) and an aprotic solvent to form a compound having the structure:

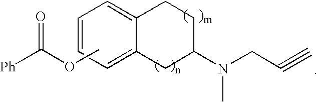

94. The process of claim 93, wherein the aprotic solvent in step (d) is CHCl₃.

95. A process for preparing a compound having the structure: 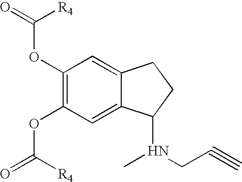
wherein R₄ is branched or unbranched C₁ to C₆ alkyl, aryl, aralkyl or NR₅R₆,
wherein R₅ and R₆ are each independently H, C₁ to C₈ alkyl, C₆ to C₁₂ aryl, C₆ to C₁₂ aralkyl or C₆ to C₁₂ cycloalkyl, each optionally substituted;
which process comprises:
(a) reacting a compound having the structure: 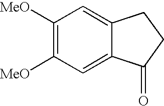
with AlCl₃ or BBr₃ in the presence of toluene to produce a compound having the structure: 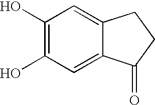
(b) reacting the product formed in step (a) with benzyl chloride and K₂CO₃ in the presence of dimethyl formamide (DMF) to produce a compound having the structure: 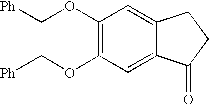
(c) reacting the product formed in step (b) with MeNH₂.HCl, NaCNBH₃ in tetrahydrofuran (THF)/MeOH to produce a compound having the structure: 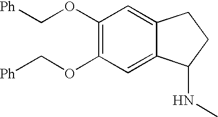
(d) reacting the product formed in step (c) with H₂, Pd/C and MeOH to produce a compound having the structure: 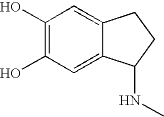
(e) reacting the product formed in step (d) with Boc₂O, dioxane/H₂O and NaHCO₃ to produce a compound having the structure: 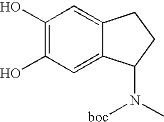
(f) reacting the product formed in step (e) with R₄COCl, Et₃N in CH₂Cl₂ in the presence of 4-dimethylaminopyridine (DMAP) to produce a compound having the structure: 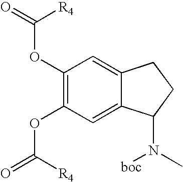
(g) reacting the product formed in step (f) with HCl/dioxane to produce a compound having the structure: 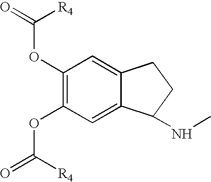
(h) reacting the product formed in step (g) with propargyl bromide, K₂CO₃ in CH₃CN and then with HCl/ether and MeOH to produce a compound having the structure:

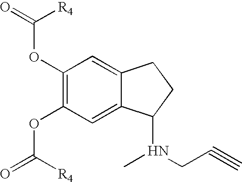

96. The process of claim 95 for preparing a compound having the structure: 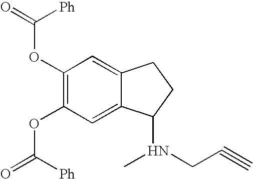
which comprises:
(a) reacting a compound having the structure: 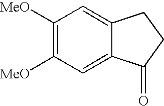
with AlCl₃ or BBr₃ in the presence of toluene to produce a compound having the structure: 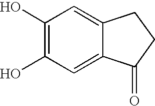
(b) reacting the product formed in step (a) with benzyl chloride and K₂CO₃ in the presence of dimethyl formamide (DMF) to produce a compound having the structure: 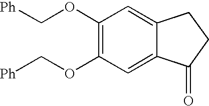
(c) reacting the product formed in step (b) with MeNH₂.HCl, NaCNBH₃ in tetrahydrofuran (THF)/MeOH to produce a compound having the structure: 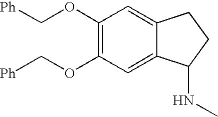
(d) reacting the product formed in step (c) with H₂, Pd/C and MeOH to produce a compound having the structure: 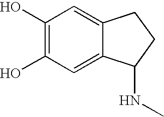
(e) reacting the product formed in step (d) with Boc₂O, dioxane/H₂O and NaHCO₃ to produce a compound having the structure: 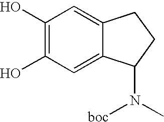
(f) reacting the product formed in step (e) with PhCOCl, Et₃N in CH₂Cl₂ in the presence of 4-dimethylaminopyridine (DMAP) to produce a compound having the structure: 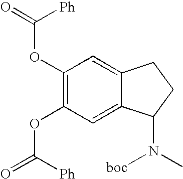
(g) reacting the product formed in step (f) with HCl/dioxane to produce a compound having the structure: 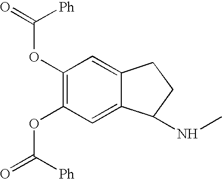
(h) reacting the product formed in step (g) with propargyl bromide, K₂CO₃ in CH₃CN and then with HCl/ether and MeOH to produce a compound having the structure:

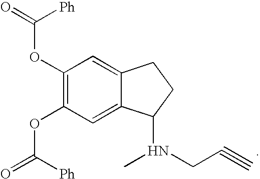

97. Use of a compound or a prodrug of a compound which becomes the compound having the structure: 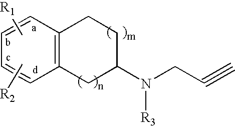
wherein R₁ is OH or OC(O)R₄;
wherein R₂ is H, OH or OC(O)R₄,
wherein R₄ is branched or unbranched C₁ to C₆ alkyl, aryl, aralkyl or NR₅R₆,
wherein R₅ and R₆are each independently H, C₁ to C₈ alkyl, C₆ to C₁₂ aryl, C₆ to C₁₂ aralkyl or C₆ to C₁₂ cycloalkyl, each optionally substituted;
wherein R₃ is H or C₁ to C₆ alkyl;
wherein n is 0 or 1; and
wherein m is 1 or 2,
or a pharmaceutically acceptable salt thereof, for the manufacture of a medicament for treating a subject afflicted with a neurological disease, wherein the compound is to be periodically administered to the subject in a therapeutically effective dose.

98. Use of a compound or a prodrug of a compound which becomes the compound having the structure: 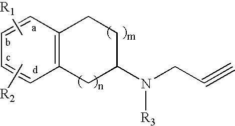
wherein R₁ is OH or OC(O)R₉, and
wherein R₉ is branched or unbranched C₁ to C₆ alkyl, aryl, or aralkyl;
R₂ is H or OC(O)R₄, or both R₁ and R₂ are OC(O)R₄,
wherein R₄ is branched or unbranched C₁ to C₆ alkyl, aryl, aralkyl or NR₅R₆,
wherein R₅ and R₆ are each independently H, C₁ to C₈ alkyl, C₆ to C₁₂ aryl, C₆ to C₁₂ aralkyl or C₆ to C₁₂ cycloalkyl, each optionally substituted;
wherein R₃ is H or C₁ to C₆ alkyl;
wherein n is 0 or 1; and
wherein m is 1 or 2,
or a pharmaceutically acceptable salt thereof, for the manufacture of a medicament for treating neurological disease in a subject, wherein the compound is to be periodically administered to the subject in a therapeutically effective dose.

99. The use of claim 98, wherein the compound has the structure: 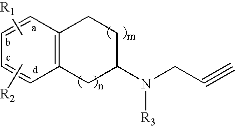
wherein R₁ is OC(O)R₉ and R₂ is H,
wherein R₉ is branched or unbranched C₁ to C₆ alkyl, aryl, or aralkyl, or
R₁ is OC (O)R₄ and R₂ is OC(O) R₄,
wherein R₄ is branched or unbranched C₁ to C₆ alkyl, aryl, aralkyl or NR₅R₆,
wherein R₅ and R₆are each independently H, C₁ to C₈ alkyl, C₆ to C₁₂ aryl, C₆ to C₁₂ aralkyl or C₆ to C₁₂ cycloalkyl, each optionally substituted;
wherein R₃ is H or C₁ to C₆ alkyl;
wherein n is 0 or 1; and
wherein m is 1 or 2.

100. The use of claim 98, wherein the compound has the structure: 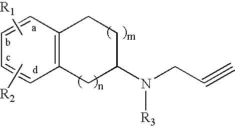
wherein R₁ is OH;
wherein R₂ is H or OC(O)R₄ when R₁ is attached to the “a” carbon or the “d” carbon, or
R₂ is OC(O)R₄ when R₁ is attached to the “b” carbon or the “c” carbon;
wherein R₄ is C₁ to C₆ branched or unbranched alkyl, aryl, aralkyl or NR₅R₆,
wherein R₅ and R₆are each independently H, C₁ to C₈ alkyl, C₆ to C₁₂ aryl, C₆ to C₁₂ aralkyl or C₆ to C₁₂ cycloalkyl, each optionally substituted;
wherein R₃ is H or C₁ to C₆ alkyl;
wherein n is 0 or 1; and
wherein m is 1 or 2.

101. The use of claim 98, wherein the compound has the structure: 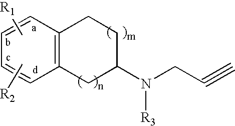
wherein the compound is an optically pure enantiomer;
wherein R₁ is OH;
wherein R₂ is H;
wherein R₃ is H or C₁ to C₆ alkyl;
wherein n is 0 or 1; and
wherein m is 1 or 2.

102. The use of claim 98, wherein the subject is human.

103. The use of claim 98, wherein the medicament is formulated for oral, parenteral, intravenous, transdermal, or rectal administration.

104. The use of claim 98, wherein the therapeutically effective amount is from about 0.01 mg per day to about 50.0 mg per day.

105. The use of claim 98, wherein the therapeutically effective amount is from about 0.1 mg per day to about 100.0 mg per day.

106. The use of claim 105, wherein the therapeutically effective amount is from about 0.1 mg per day to about 10.0 mg per day.

107. The use of claim 98, wherein the neurological disease is Parkinson's disease, Alzheimer's disease, depression, epilepsy, narcolepsy, amyotrophic lateral sclerosis (ALS), memory disorders, panic, post-traumatic stress disorder (PTSD), sexual dysfunction, attention deficit and hyperactivity syndrome (ADHD), attention deficit disorder, or Tourette's syndrome.

108. The use of claim 107, wherein the neurological disease is depression.

109. The use of claim 108, wherein the compound has the structure: [chemical structure]
